(19) United States
(12) Patent Application Publication
(10) Pub. No.: US 2003/0040029 A1
(43) Pub. Date: Feb. 27, 2003

(54) Detection of tumor marker transcript and protein recognized by naive natural killer cells

(76) Inventor: Allen J. Norin, Pomona, NY (US)

Correspondence Address: HOFFMANN & BARON, LLP, 6900 JERICHO TURNPIKE, SYOSSET, NY, 11791, US

(21) Appl. No.: 09/896,908

(22) Filed: Jun. 29, 2001

Related U.S. Application Data
(63) Continuation-in-part of application No. 09/423,585, filed on Nov. 9, 1999, now abandoned.
Application No. 09/423,585 is a 371 of international application No. PCT/US98/10021, filed on May 15, 1998.
(60) Provisional application No. 60/046,553, filed on May 15, 1997.

(51) Int. Cl. G01N 33/574; C12N 9/00; C07K 16/46

(52) U.S. Cl. 435/7.23; 435/188.5; 530/391.1

(57) ABSTRACT

The invention provides methods for detection, diagnosis and monitoring of tumor cells in a sample of mammalian cells. In one aspect, the invention provides methods for detecting the protein, p38.5 expressed at the cell surface, where cell surface expression of p38.5 is indicative of a tumor cell, particularly of hematopoietic origin. In another aspect the invention provides methods of diagnosis of tumor cells by determining the total level of expression of p38.5 or p38.5-specific RNA, a higher than normal level of total p38.5 expression by the cells being diagnostic of tumor cells in a wide range of cell types. Methods of treating tumors expressing cell surface p38.5 are also provided. Also described are screening methods for identifying compounds that inhibit NK mediated cytotoxicity.

This application is a continuation in part of U.S. Ser. No. 09/423,585 which was filed on Nov. 9, 1999, which claims the benefit of International Patent Application PCT/US98/10021, filed on May 15, 1998, which claims the benefit of U.S. Provisional Application No. 60/046,553, filed May 15, 1997.

This invention was made with government support under Grant No. RO1 CA47548 from the National Institutes of Health. The government may have certain rights in the invention. The amplified product is

### FIELD OF THE INVENTION

The present invention relates to a tumor marker transcript and its protein product found in tumor cells, including those susceptible to cell-mediated cytotoxic killing by naïve Natural Killer (NK) cells. Further, the invention relates to methods of detection and diagnosis of tumors as well as therapeutic treatments and methods of monitoring treatment regimens.

### BACKGROUND OF THE INVENTION

Discovery of genes that are differentially expressed in malignant tissues provides potential new targets for diagnosis and therapy of cancerous diseases. Numerous markers of malignant diseases have been identified since the discovery of carcinoembryonic antigen (CEA), Gold, P. and Freedman, S. J Exp Med, 121, 439-459 (1965). Some of these markers are associated with organ specific cancers whereas others have a more extensive tissue distribution (Bast, R. et al. N Engl J Med, 309, 883-887 (1983) discloses CA125 as a common marker in ovarian cancer; Chodosh, L., J Mammary Gland Biol Neoplasia, 3, 389-402 (1998) disclose expression patterns of BRCA1 and BRCA2 in normal and neoplastic cells; Gregory, J. J. and Finlay, J. Drugs, 57, 463-467 (1999) disclose alpha-fetoprotein and beta-human chorionic gonadotropin as tumor markers.; Kloppel, G. and Caselitz, J. Epithelial tumor markers: oncofetal antigens (carcinoembryonic antigen, alpha fetoprotein) and epithelial membrane antigen., p. 103-132, Berlin (1987); Kos, J. et al. Int J Biol Markers, 15, 84-89 (2000) disclose the use of the cysteine proteinases, cathepsins and their inhibitors as markers for diagnosis and prognosis in cancer.; Lapidus, R., et al. J Mammary Gland Biol Neoplasia, 3, 85-94 (1998) disclose loss of estrogen and progesterone receptor gene expression in human breast cancer; Ordonez, N., Adv Anat Pathol, 7, 123-127 (2000) disclose thyroid transcription factor-1 is a marker of lung and thyroid cancinomas; Sahin, A. Adv Anat Pathol, 7, 158-166 (2000) disclose amplification of the HER-2/neu (cerbB-2) gene commonly found in breast cancer; Wang, M., et al. Invest Urol, 17, 159-163 (1979) discloses detection of human prostate specific antigen in normal, benign hypertrophic and malignant prostatic tissues.

Many normal gene products are either over-expressed in cancerous diseases e.g., PSA, Ca125 (Chang, S., et al. Mol Urol, 3, 313-320, 1993) and/or are translocated to abnormal sites within malignant cells. BRCA1, which is expressed at normal levels in breast cancer is translocated to an abnormal cellular site (Chen, Y. et al. Science, 270, 789-791, 1995). The wild type BRCA I gene product normally localizes to the nucleus via two putative signal sequences (nuclear localization sequences, NLSs) that are similar to those found in steroid hormone receptor molecules (Boulikas, T. J Cell Biochem, 55, 32-58, 1994). Mutant forms of BRCA I that occur in breast and ovarian cancer localize, however, to the cytoplasm (Chen et al., 1995 id). Similar mis-transport has been demonstrated for transcription factors involved in oncogenesis such as c-Fos (Roux, P. et al. Cell, 63, 341-351, 1990) and SV40 large T antigen (Schneider, J., et al. Cell, 54, 117-125, 1988).

Tumor cells may be recognized and killed by activated natural killer (NK) cells or in certain cases, by naïve NK cells. Naïve NK cells (CD3⁻, CD16⁺ TCR⁻), unlike cytolytic T lymphocytes (CTL) (CD3⁺, CD16⁻, TCR⁺) provide cell-mediated lytic activity against virus infected cells and certain tumors without requirement for activation. See Trinchieri, G. Adv. Immunol. 47:187-376 1989; Whiteside, T. L. and R. B. Herberman Immunol. Allergy Clin. N. Amer. 10:663-704 (1990); Bancroft, G. J. Curr. Opin. Immunol. 5:503-510 (1993). Such unactivated lymphocytes, also referred to as resting or naïve NK cells, are capable of destroying a narrow spectrum of tumor cells. See Trinchieri, G. (1989) id; Whiteside T. L., in Current Protocols in Immunology, J. E. Coligan et al. eds., Supplement 17, Unit 7-18, John Wiley & Sons, New York (1996). Upon activation with lymphokines such as IL-2, NK cells acquire broad anti-tumor lytic activity, becoming lymphokine activated killer cells (LAK cells). The mechanism(s) by which naïve NK cells recognize their target cells is not completely understood. Interaction of cellular adhesion molecules and recognition of specific target structure(s) have been proposed as critical initial events in the cell mediated lytic process. Trinchieri, G. (1989) id; Storkus, W. J. and J. R. Dawson Critical Rev. Immunol. 10:393-416 (1989). It is well known, for example, that the initiation of target cell lysis by CTL is due to interaction of the major histocompatibility complex (MHC) class I molecules (plus bound peptide) with the T cell receptor (TCR). Davis, M. M. and P. J. Bjorkman. Nature (Lond.) 334:395-401 (1988); Townsend, A. and H. Bodmer Ann. Rev. Immunol. 7:601-624 (1989). Analogous molecular structures that initiate the lytic process between NK cells and tumor cells have not been defined. Although MHC molecules may serve a regulatory function for NK cells, see for example Gumperz, J. E. and P. Perham Nature (Lond.) 378:245-248 (1995); Yokoyama, W. M. Proc. Natl. Acad. Sci. (USA) 92:3081-3085 (1995); Lanier, L. L. and J. H. Phillips Immunol. Today 17:86-91 (1996); Moretta, A. et al. J. Exp. Med. 182:875-884 (1995), it is clear that their presence on the surface of tumor cells is not required for cytolysis since many NK susceptible cell lines do not express MHC gene products. Moretta, A., et al. 1995, id; Litwin, V. et al. J. Exp. Med. 178:1321-1336 (1993).

Previous attempts during the past two decades to identify recognition structures exclusive to NK cell-tumor interaction have been unsuccessful (Gumperz, J. E. and P. Perham (1995) id., though important components on both NK cells and on tumor cells that contribute to cellular adhesion and regulation of the cytolytic process have been revealed. These receptor-ligand interactions, however, are not unique to NK cells since they also occur between T lymphocytes and their respective target cells (Lanier, L. L. and J. H. Phillips Immunol. Today 17:86-91 (1996). The surface proteins responsible for NK cell specific receptor-ligand interactions still remain largely unknown.

Many forms of cancer are known and accordingly, there is a continuing need for new tumor marker molecules. Moreover, there is a need in the art for new methods that detect and accurately determine the level of such markers in cells, and for therapeutic methods to treat mammals, particularly humans suffering from these cancers.

### SUMMARY OF THE INVENTION

The present invention provides methods of detection of a protein designated as “p38.5” having a molecular weight of about 38.5 kD as a tumor cell marker which binds natural killer (NK) cells and which is found to be expressed intracellularly in a wide variety of tumor cells. Furthermore, p38.5 is characteristically expressed on the surface of tumor cells susceptible to cell-mediated lysis by naïve natural killer (NK) cells.

The invention provides a method for diagnosing tumor cells. The method includes obtaining a cell sample from a subject, and assessing the level of p38.5 present in the cell sample. Detection of a higher than normal level of p38.5 in the cell is indicative of a tumor cell.

The invention also provides another method for diagnosing tumor cells. The method includes obtaining a cell sample from a subject, and assessing the level of p38.5-specific RNA present in the cell sample. Detection of a higher than normal level of p38.5-specific RNA in the cell is indicative of a tumor cell.

The invention provides a method of monitoring the progress of a disorder, disease or condition associated with a higher than normal level of expression of p38.5 in a subject. The method includes obtaining two or more cell samples from the subject at different times and assessing the level of p38.5 expressed at the surface of the cells in each of the cell samples; or assessing the total p38.5 expressed by the cells in each of the cell samples. Comparison of the level of p38.5 expressed at the surface of the cells in each of the cell samples or of the total p38.5 expressed by the cells in each of the cell samples permits monitoring of the progress of the disorder, disease or condition.

The invention provides another method of monitoring the progress of a disorder, disease or condition associated with a higher than normal level of expression of p38.5 in a subject. The method includes obtaining two or more cell samples from the subject at different times and assessing level of p38.5-specific RNA present in each of the cell samples. Comparison of the level of p38.5-specific RNA present in each of the cell samples permitting monitoring of the progress of the disorder, disease or condition.

The invention further provides a method for treating a subject suffering from a tumor associated with cell surface expression of p38.5. The method includes administering to the subject an effective amount of an antibody that specifically binds p38.5.

The invention also provides a method for identifying a compound that inhibits NK cell mediated killing of cells that express a higher than normal level of cell surface p38.5. The method includes providing a first sample of cells that expresses a higher than normal level of cell surface p38.5 and contacting the first sample of cells with the compound to be tested. The first sample of cells is then exposed to naïve NK cells and the level of cytotoxicity is assessed. A second sample of cells identical to the first sample is provided and is exposed to naïve NK cells and the level of cytotoxicity is assessed. The NK cell mediated cytotoxicity in the first sample of cells is compared with the NK cell mediated cytotoxicity in the second sample of cells. A reduction in the level of cytotoxicity by the compound identifies the compound as an inhibitor of natural killer (NK) cell mediated killing of cells that express p38.5 at their cell surface.

The invention further provides a method for determining the number of natural killer (NK) cells in a biological sample. The method includes: contacting the sample containing NK cells with an antibody specifically binds p70 protein, under conditions permissive for binding of the antibody to p70 protein expressed by NK cells in said sample; and counting the number of cells bound by the antibody; thereby permitting determination the number of natural killer cells in the sample.

These and other advantages of the invention will be appreciated from the detailed description and examples which are set forth herein. The detailed description and examples enhance the understanding of the invention, but are not intended to limit the scope of the invention.

### BRIEF DESCRIPTION OF THE DRAWINGS

FIG. 1 is a photograph of Western blots demonstrating the Binding of K562 membrane proteins to subsets of Human peripheral blood lymphocytes (HPBL).

FIG. 2 is a photograph of Western blots demonstrating the binding of purified p38.5 to HPBL.

FIG. 3A to FIG. 3F are plots from flow cytometry analysis of anti-p38.5 treated NK-susceptible and NK-resistant tumor cell lines.

FIG. 3G is a photograph of Western blots of anti-p38.5 immunoprecipitates from surface labeled cells.

FIG. 4 is a bar graph illustrating the relationship between cell surface expression of p38.5 on normal and variant tumor cell lines and susceptibility of tumors to NK cell mediated lysis.

FIG. 5 is a bar graph illustrating the effect of purified K562 membrane proteins on naïve NK cell-mediated cytotoxicity.

FIG. 6 is a Northen blot of p38.5 gene transcripts in malignant and non-malignant cells.

FIG. 7 is an agarose gel resolution of RT-PCR products of p38.5 mRNA from malignant and non-malignant cells.

FIG. 8 is a Western blot of p38.5 detected in malignant and non-malignant cells.

FIG. 9A is a Western blot analysis of p38.5 and F₁β-ATPase in whole cell extracts and particulate fractions of malignant and non-malignant cells.

FIG. 9B is a semi-quantitative analysis of RT-PCR products of p38.5 gene transcripts in a malignant and non-malignant cell line.

FIG. 10 is an agarose gel resolution of RT-PCR products of total RNA from T cells activated with PMA or PHA.

### DETAILED DESCRIPTION OF THE PREFERRED EMBODIMENTS

A protein having a molecular weight of about 38.5 kilodaltons (kD) (hereinafter designated “p38.5”), has been found to be characteristically expressed on the surface of tumor cells that are susceptible to cell-mediated lysis by naïve (non-activated) human natural killer (NK) cells. Further, this protein now has been shown to be present intracellularly at higher than normal levels in a variety of tumor lines from different tissues. The level of expression of p38.5 was found to be significantly lower in normal cells and non-malignant cell lines than the levels found in tumor cells when equivalent cell numbers were assayed.

Analysis of p38.5 and p38.5-specific mRNA levels demonstrated that p38.5 is highly expressed in a number of representative malignant cell lines of different tissue origins. In contrast, non-malignant cell lines and normal cells expressed substantially lower levels of the protein and p38.5-specific mRNA. The concordance of elevated levels of both protein and specific mRNA in malignant cell lines compared to non-malignant cells suggests transcriptional up-regulation of the p38.5 gene in cancer cells. Since non-malignant as well as malignant cell lines are immortal and have been maintained in long term culture it is unlikely that these latter conditions are sufficient to induce high level of p38.5 gene expression in malignant cell lines. Over-expression of the p38.5 gene in vivo, therefore, serves as a marker of a cancerous disease, disorder or condition.

p38.5 binds preferentially to NK cells in the presence of other types of lymphocytes. Lymphocytes include, in general B cells, T cells. As a result, p38.5 may be used in methods of selective detection, identification, and separation of NK cells in biological samples, such as blood samples.

The term “natural killer (NK) cells” as used herein, refers to non-T, non-B lymphocytes that are defined by the following properties: They have spontaneous lytic activity against cells infected with intra-cellular parasites (e.g. viruses) and certain types of tumor cells (usually of hematologic origin). They express various combinations of CD8, CD16 and CD56 on their surface and lack the surface expression of CD3 components, TCR heterodimers and immunoglobulins (e.g., IgM, IgD). They express cell surface molecules that regulate cytolytic activity by interaction with MHC class-1 molecules. They may be stimulated by incubation with cytokines, e.g. IL-2, to express lytic activity against a broader spectrum of target cells.

The property of sensitivity of a tumor cell to killing by naïve NK cells is imparted by p38.5 at the surface of the tumor cell. The p38.5 may be isolated from tumor cells that are susceptible to cell-mediated lysis by naïve NK cells or from tumor cells that express p38.5 intracellularly.

In general, tumors or neoplasms include new growths of tissue in which the multiplication of cells is uncontrolled and progressive. Some such growths are benign, but others are termed “malignant,” leading to death of the organism. Malignant neoplasms or “cancers” are distinguished from benign growths in that, in addition to exhibiting aggressive cellular proliferation, they invade surrounding tissues and metastasize. Moreover, malignant neoplasms are characterized in that they show a greater loss of differentiation (greater “dedifferentiation”), and of their organization relative to one another and their surrounding tissues. This property is also called “anaplasia.”

Tumor cells susceptible to cell-mediated lysis by non-activated NK cells typically include hematologic tumors and the like. For example, leukemias. Leukemias include tumors derived from blood cell-forming tissues and include acute and chronic forms, which may be myeloid, lymphatic, or monocytic varieties. Other types of tumors, such as carcinomas, adenocarcinomas, and sarcomas are ordinarily resistant to killing by NK cells. Carcinomas include those malignant neoplasms derived from epithelial cells which tend to infiltrate (invade) the surrounding tissues and give rise to metastases. Adenocarcinomas are carcinomas derived from glandular tissue or in which the tumor cells form recognizable glandular structures. Sarcomas broadly include tumors whose cells are embedded in a fibrillar or homogeneous substance like embryonic connective tissue.

The invention is particularly illustrated herein by reference to treatment of certain types of experimentally defined cancers. In these exemplified treatments, standard state-of-the-art in vitro models have been used. For example, K562 (erythroleukemia) cells, MOLT-4 (T cell leukemia) cells, and Jurkat (T cell lymphoma) cells are all examples of well defined NK-susceptible tumor lines. These methods may be used to identify agents that may be expected to be efficacious in in vivo treatment regimens. However, it will be understood that the method of the invention is not limited to the treatment of these tumor types, but extends to any tumor that expresses p38.5 at a higher than normal level, especially NK-susceptible tumors. The tumors may be derived from any tissue or organ system.

The normal level of expression of p38.5 or cell surface p38.5 in any particular cell type from a particular tissue may be routinely determined by anyone of skill in the art according to the methods taught herein. Cell samples from normal tissues are assayed by the methods of the invention and a normal level of expression of p38.5 is established and the normal range of variation of this level will be evident from the variation among random normal samples. Similarly the normal level and variation in expression of p38.5-specific RNA may be routinely determined by the nucleic acid methods of the invention.

A higher than normal level of expression of p38.5 or cell surface p38.5 is recognized by the skilled artisan as a level of expression of p38.5 or cell surface p38.5 that is higher than the determined normal level. Similarly, A higher than normal level of expression of p38.5-specific RNA is recognized as a level of expression of p38.5-specific RNA that is higher than the determined normal level.

cDNA and Deduced Amino Acid Sequences of Human and Mouse p38.5

The nucleotide sequences of several cDNA clones obtained as detailed below in Example 20 were confirmed by sequencing overlapping clones. Overlapping clones 70 and 402 provide the nucleotide sequence shown in Table 1. This cDNA sequence is believed to encode the entire human p38.5 (shown in Table 2).

TABLE 1

Nucleotide Sequence of cDNA (SEQ ID NO:1) encoding human p
38.5

| | |
|---|---|
| −10 | GCAGGCGACC |
| 1 | ATGGGGAACG TGTTGGCTGC CAGCTCGCCG CCCGCAGGGC CGCCACCGCC |
| 51 | GCCTGCGCCG GCCCTCGTGG GGCTGCCGCC ACCTCCGCCC TCGCCGCCGG |
| 101 | GCTTCACGCT GCCGCCGCTG GGAGGCAGCC TGGGCGCCGG ACCAGTACGA |
| 151 | GTCGAAGTTC GGAACGGACC CCCGGGGCTG CAACCGCCAG CGCCTCAGGG |
| 201 | GCCGCCGAGG ATGGGGCCTG CGGCTGCCTG CCCAACCCGG GCACATTCGA |
| 251 | GGAGTGCCAC CGGAAGTGCA AGGAGCTGTT TCCCATTCAG ATGGAGGGTG |
| 301 | TCAAGCTCAC AGTCAACAAA GGGTTGAGTA ACCATTTTCA GGTCAACCAC |
| 351 | ACAGTAGCCC TCAGCACAAT CGGGGAGTCC AACTACCACT TCGGGGTCAC |
| 401 | ATATGTGGGG ACAAAGCAGC TGAGTCCCAC AGAGGCGTTC CCTGTACTGG |
| 451 | TGGGTGACAT GGACAACAGT GGCAGTCTCA ACGCTCAGGT CATTCACCAG |
| 501 | CTGGGCCCCG GTCTCAGGTC CAAGATGGCC ATCCAGACCC AGCAGTCGAA |
| 551 | GTTTGTGAAC TGGCAGGTGG ACGGGGAGTA TCGGGGCTCT GACTTCACAG |
| 601 | CAGCCGTCAC CCTGGGGAAC CCAGACGTCC TCGTGGGTTC AGGAATCCTC |
| 651 | GTAGCCCACT ACCTCCAGAG CATCACGCCT TGCCTGGCCC TGGGTGGAGA |
| 701 | GCTGGTCTAC CACCGGCGGC CTGGAGAGGA GGGCACTGTC ATGTCTCTAG |
| 751 | CTGGGAAATA CACATTGAAC AACTGGTTGG CAACGGTAAC GTTGGGCCAG |
| 801 | GCGGGCATGC ACGCAACATA CTACCACAAA GCCAGTGACC AGCTGCAGGT |
| 851 | GGGTGTGGAG TTTGAGGCCA GCACAAGGAT GCAGGACACC AGCGTCTCCT |
| 901 | TCGGGTACCA GCTGGACCTG CCCAAGGCCA ACCTCCTCTT CAAAGGCTCT |
| 951 | GTGGATAGCA ACTGGATCGT GGGTGCCACG CTGGAGAAGA AGCTCCCACC |
| 1001 | CCTGCCCCTG ACACTGGCCC TTGGGGCCTT CCTGAATCAC CGCAAGAACA |
| 1051 | AGTTTCAGTG TGGCTTTGGC CTCACCATCG GCTGAGCCCT CCTGGCCCCC |
| 1101 | GCCTTCCACG CCCTTCCGAT TCCACCTCCA CCTCCACCTC CCCCTGCCAC |
| 1151 | AGAGGGGAGA CCTGAGCCCC CCTCCCTTCC CTCCCCCCTT GGGGGTCGGG |
| 1201 | GGGGGACATT GGAAAGGAGG GACCCCGCCA CCCCAGCAGC TGAGGAGGGG |
| 1251 | ATTCTGGAAC TGAATGGCGC TTCGGGATTC TGAGTAGCAG GGGCAGCATG |
| 1301 | CCCAGTGGGC CTGGGGTCCC GGGAGGGATT CCGGAATTGA GGGGCACGCA |
| 1351 | GGATTCTGAG CACCAGGGGC AGAGGCGGCC AGACAACCTC AGGGAGGAGT |
| 1401 | GTCCTGGCGT CCCCATCCTC CAAAGGGCCT GGGCCCGCCC CGAGGGGGCA |
| 1451 | GCGAGAGGAG CTTCCCCATC CCCGGTCAGT CCACCCTGCC CCGTCCACTT |
| 1501 | TCCCATCTCC TCGGTATAAA TCATGTTTAT AAGTTATGGA AGAACCGGGA |
| 1551 | CATTTTACAG AAAAAAAACA AAAAACAACA AAAAATATAC GTGGGAAAAA |
| 1601 | AAAAAAAAAA AAAAAAAAAA AAAAAAAAAA AAAAAAA |

Clone 70 was selected from an expression vector library containing K562 cDNA; clone 402 was generated by RT-PCR using K562 total RNA and the following forward and reverse primers (their target seqences are shown in bold in the Table):
EC4, GCA GGC GAC CAT GGG GAA CGT (SEQ ID NO:3),
LP3, TGT CCC GGT CCT TCC ATA AC (SEQ ID NO:4).
See Example 20.
The nucleotides are numbered beginning from the A of the ATG start codon (underlined) to the TGA stop codon (also underlined) at 1083-1085.
Note: primer LP3 was found to have a single base mismatch at nucleotide 1543 (mis-match is at base 10, which is a C in the primer (underlined). The target nucleotide at position 1543 is an A.
The positions of primer pairs (bolded in the above sequence) used in polymerase chain reactions were as follows:
Forward Primers: EC4 = −10 to 11; LP1 = 383-402; RS1 = 531-551.
Reverse Primers: RS1 = 1287-1267; XF2 = 1399-1370; LP3 = 1552-1533.

TABLE 2

Complete amino acid sequence encoded in the ORF of human p38.5
cDNA. (SEQ ID NO:2).

```
Met Gly Asn Val Leu Ala Ala Ser Ser Pro Pro Ala Gly Pro Pro Pro
1 5 10 15
Pro Pro Ala Pro Ala Leu Val Gly Leu Pro Pro Pro Pro Pro Ser Pro
                        20 25 30
Pro Gly Phe Thr Leu Pro Pro Leu Gly Gly Ser Leu Gly Ala Gly Thr
                                                        40 45
Ser Thr Ser Arg Ser Ser Glu Arg Thr Pro Gly Ala Ala Thr Ala Ser
        50 55 60
Ala Ser Gly Ala Ala Glu Asp Gly Ala Cys Gly Cys Leu Pro Asn Pro
65 70 75 80
Gly Thr Phe Glu Glu Cys His Arg Lys Cys Lys Glu Leu Phe Pro Ile
                                85 90 95
Gln Met Glu Gly Val Lys Leu Thr Val Asn Lys Gly Leu Ser Asn His
                        100 105 110
Phe Gln Val Asn His Thr Val Ala Leu Ser Thr Ile Gly Glu Ser Asn
                115 120 125
Tyr His Phe Gly Val Thr Tyr Val Gly Thr Lys Gln Leu Ser Pro Thr
        130 135 140
Glu Ala Phe Pro Val Leu Val Gly Asp Met Asp Asn Ser Gly Ser Leu
145 150 155 160
Asn Ala Gln Val Ile His Gln Leu Gly Pro Gly Leu Arg Ser Lys Met
                                165 170 175
Ala Ile Gln Thr Gln Gln Ser Lys Phe Val Asn Trp Gln Val Asp Gly
                        180 185 190
Glu Tyr Arg Gly Ser Asp Phe Thr Ala Ala Val Thr Leu Gly Asn Pro
                195 200 205
Asp Val Leu Val Gly Ser Gly Ile Leu Val Ala Arg Tyr Leu Gln Ser
        210 215 220
Ile Thr Pro Cys Leu Ala Leu Gly Gly Glu Leu Val Tyr His Arg Arg
225 230 235 240
Pro Gly Glu Glu Gly Thr Val Met Ser Leu Ala Gly Lys Tyr Thr Leu
                                245 250 255
Asn Asn Trp Leu Ala Thr Val Thr Leu Gly Gln Ala Gly Met His Ala
                        260 265 270
Thr Tyr Tyr His Lys Ala Ser Asp Gln Leu Gln Val Gly Val Glu Phe
                275 280 285
Glu Ala Ser Thr Arg Met Gln Asp Thr Ser Val Ser Phe Gly Tyr Gln
        290 295 300
Leu Asp Leu Pro Lys Ala Asn Leu Phe Phe Lys Gly Ser Val Asp Ser
305 310 315 320
Asn Trp Ile Val Gly Ala Thr Leu Glu Lys Lys Leu Pro Pro Leu Pro
                                325 330 335
Leu Thr Leu Ala Leu Gly Ala Phe Leu Asn His Arg Lys Asn Lys Phe
                        340 345 350
Gln Cys Gly Phe Gly Leu Thr Ile Gly
                355 360
```

The amino acid sequence from Phe185 to Arg195 corresponds to the 11-mer peptide originally isolated from the purified native protein. See Example 11.
The 25-mer-peptide shown in bold type at Ser43 to Gly67 was initially deduced from clone 5′R obtained by 5′RACE. See Example 19.

TABLE 3

Alignment of p38.5 encoding cDNAs from mouse (SEQ ID NO:5)
and human (SEQ ID NO:1)

| | |
|---|---|
| | 10 20 30 40 50 |
| human | ATGGGGAACGTGTTGGCTGCCAGCTCGCCGCCCGCAGGGCCGCCACCGCC |
| | :::::::::::::::::::::::::: :::::::::::::: :: ::::: |
| mouse | ATGGGGAACGTGTTGGCTGCCAGCTCTCCGCCCGCAGGGCCACCGCCGCC |
| | 10 20 30 40 50 |
| | 60 70 80 90 100 |
| human | GCCTGCGCCGGCCCTCGTGGGGCTGCCGCCACCTCCGCCCTCGCCGCCGG |
| | :: ::::: :::::::::::::: ::::: :: :: :: :::::::: : |
| mouse | TCCTACGCCGTCCCTCGTGGGGCTCCCGCCGCCGCCTCCTTCGCCGCCAG |
| | 60 70 80 90 100 |
| | 110 120 130 140 |
| human | GCTTCACGCTGCCGCCGCTGGGAGGCAGCCTGGGCGCCGGACCA-GTACG |
| | ::::::: ::::::::::: :: ::: :::::::: : :: :: ::: |
| mouse | GCTTCACTCTGCCGCCGCTCGGCGGCGGCCTGGGCACTGGGTCAAGCACT |
| | 110 120 130 140 150 |
| | 150 160 170 180 190 |
| human | AGTCGAAGTTCGGAACGGACCCCCGGGGCTGCAACCGCCAGCGCCTCAGG |
| | : :: ::::::::::::: ::::::::::: :: : :::: : : |
| mouse | GGCCGTGGTTCGGAACGGACTCCCGGGGCTGCGGCCAGCGGCGCTGCGGC |
| | 160 170 180 190 200 |
| | 200 210 220 230 240 |
| human | GGCCGCCGAGGATGGGGCCTGCGGCTGCCTGCCCAACCCGGGCACATTCG |
| | :::: : :: :::::: :::::: ::::::::::::::::: :: :::: |
| | 210 220 230 240 250 |
| | 250 260 270 280 290 |
| human | AGGAGTGCCACCGGAAGTGCAAGGAGCTGTTTCCCATTCAGATGGAGGGT |
| | :::::::::::::::::::::::::::::::::: :::::::::: ::: |
| mouse | AGGAGTGCCACCGGAAGTGCAAGGAGCTGTTTCCAGTTCAGATGGAAGGT |
| | 260 270 280 290 300 |
| | 300 310 320 330 340 |
| human | GTCAAGCTCACAGTCAACAAAGGGTTGAGTAACCATTTTCAGGTCAACCA |
| | ::::: :: :::::::::::::::::::: :::: ::: ::::: : ::: |
| mouse | GTCAAACTTACAGTCAACAAAGGGTTGAGCAACCGTTTCCAGGTGACCCA |
| | 310 320 330 340 350 |
| | 350 360 370 380 390 |
| human | CACAGTAGCCCTCAGCACAATCGGGGAGTCCAACTACCACTTCGGGGTCA |
| | ::::::::::::: ::::::: :::: ::::::::::::::: ::::::: |
| mouse | CACAGTAGCCCTCGGCACAATTGGGGGGTCCAACTACCACTTTGGGGTCA |
| | 360 370 380 390 400 |
| | 400 410 420 430 440 |
| human | CATATGTGGGGACAAAGCAGCTGAGTCCCACAGAGGCGTTCCCTGTACTG |
| | :::: :::::::: ::::::::::::::::::::::::::::: :: ::: |
| mouse | CATACGTGGGGACGAAGCAGCTGAGTCCCACAGAGGCGTTCCCCGTGCTG |
| | 410 420 430 440 450 |
| | 450 460 470 480 490 |
| human | GTGGGTGACATGGACAACAGTGGCAGTCTCAACGCTCAGGTCATTCACCA |
| | ::::::::::::::::: :::::::: ::::: :: :::::::: ::::: |
| mouse | GTGGGTGACATGGACAATAGTGGCAGCCTCAATGCACAGGTCATCCACCA |
| | 460 470 480 490 500 |
| | 500 510 520 530 540 |
| human | GCTGGGCCCCGGTCTCAGGTCCAAGATGGCCATCCAGACCCAGCAGTCGA |
| | :::: :::: :: ::::::::::: ::::::::::::::::::::::: : |
| mouse | GCTGAGCCCAGGCCTCAGGTCCAAAATGGCCATCCAGACCCAGCAGTCCA |
| | 510 520 530 540 550 |
| | 550 560 570 580 590 |
| human | AGTTTGTGAACTGGCAGGTGGACGGGGAGTATCGGGGCTCTGACTTCACA |
| | :::: ::::::::::::::::: :: ::::: :: :: :::::::::::: |
| mouse | AGTTCGTGAACTGGCAGGTGGATGGCGAGTACCGTGGTTCTGACTTCACA |
| | 560 570 580 590 600 |
| | 600 610 620 630 640 |
| human | GCAGCCGTCACCCTGGGGAACCCAGACGTCCTCGTGGGTTCAGGAATCCT |
| | :: :: ::::::::::: :::::::: ::: : ::::::::::::::::: |
| mouse | GCTGCTGTCACCCTGGGCAACCCAGATGTCTTGGTGGGTTCAGGAATCCT |
| | 610 620 630 640 650 |
| | 650 660 670 680 690 |
| human | CGTAGCCCACTACCTCCAGAGCATCACGCCTTGCCTGGCCCTGGGTGGAG |
| | ::: ::::::::::::::::::::::: :: :: :::::::::::::::: |
| mouse | CGTGGCCCACTACCTCCAGAGCATCACACCGTGTCTGGCCCTGGGTGGAG |
| | 660 670 680 690 700 |
| | 700 710 720 730 740 |
| human | AGCTGGTCTACCACCGGCGGCCTGGAGAGGAGGGCACTGTCATGTCTCTA |
| | :::: ::::::::::::::::: ::::::::::::::::::::::::::: |
| mouse | AGCTCGTCTACCACCGGCGGCCAGGAGAGGAGGGCACTGTCATGTCTCTA |
| | 710 720 730 740 750 |
| | 750 760 770 780 790 |
| human | GCTGGGAAATACACATTGAACAACTGGTTGGCAACGGTAACGTTGGGCCA |
| | ::::::::::::::: :::::::::::::::: :: :::::: ::::::: |
| _ | GCTGGGAAATACACACTGAACAACTGGTTGGCTACAGTAACGCTGGGCCA |
| | 760 770 780 790 800 |
| | 800 810 820 830 840 |
| human | GGCGGGCATGCACGCAACATACTACCACAAAGCCAGTGACCAGCTGCAGG |
| | ::: :::::::: :: :: :: :::::::::::::::::::::::::::: |
| mouse | GGCAGGCATGCATGCGACGTATTACCACAAAGCCAGTGACCAGCTGCAGG |
| | 810 820 830 840 850 |
| | 850 860 870 880 890 |
| human | TGGGTGTGGAGTTTGAGGCCAGCACAAGGATGCAGGACACCAGCGTCTCC |
| | ::::::::::::::::::::::::: ::::::::::::::::: : :::: |
| mouse | TGGGTGTGGAGTTTGAGGCCAGCACCAGGATGCAGGACACCAGTGCCTCC |
| | 860 870 880 890 900 |
| | 900 910 920 930 940 |
| human | TTCGGGTACCAGCTGGACCTGCCCAAGGCCAACCTCCTCTTCAAAGGCTC |
| | :: ::::: :::::::::::::::::::::::: :::: :: :::::::: |
| mouse | TTTGGGTATCAGCTGGACCTGCCCAAGGCCAACTTCCTTTTTAAAGGCTC |
| | 910 920 930 940 950 |
| | 950 960 970 980 990 |
| human | TGTGGATAGCAACTGGATCGTGGGTGCCACGCTGGAGAAGAAGCTCCCAC |
| | :::: : :: :::::::::::::: :::::::::::::::::::: :: : |
| mouse | TGTGAACAGTAACTGGATCGTGGGCGCCACGCTGGAGAAGAAGCTTCCGC |
| | 960 970 980 990 1000 |
| | 1000 1010 1020 1030 1040 |
| human | CCCTGCCCCTGACACTGGCCCTTGGGGCCTTCCTGAATCACCGCAAGAAC |
| | :: :::::::::::::: :::: : ::::::::::: ::::::::::: |
| mouse | CCTTGCCCCTGACACTGTCCCTCTGCGCCTTCCTGAACCACCGCAAGAAT |
| | 1010 1020 1030 1040 1050 |
| | 1050 1060 1070 1080 1090 |
| human | AAGTTTCAGTGTGGCTTTGGCCTCACCATCGGCTGAGCCCTCCTGGCCCC |
| | ::::: : ::::::::: ::::::::::::::::::::::::::: :: |
| mouse | AAGTTCCTGTGTGGCTTCGGCCTCACCATCGGCTGAGCCCTCCTGTCC-- |
| | 1060 1070 1080 1090 |
| | 1100 1110 1120 1130 1140 |
| human | CGCCTTCCACGCCCTTCCGATTCCACCTCCACCTCCACCTCCCCCTGCCA |
| | :::: :: : :: ::: |
| mouse | ----TTCC----TCTGCAGA---------------------------CCA |
| | 1100 1110 |
| | 1150 1160 1170 1180 1190 |
| human | CAGAGGGGAGACCTGAGCCCCCCTCCCTTCCCTCCCCCCTTGGGGGTCGG |
| | : ::: ::: : ::::::: :::::: : : : ::::: :: |
| mouse | TCGCTGGGCCGGCTGCCCTCCCCTCCTCTCCCTCTCTCTT--GGGGTTGG |
| | 1120 1130 1140 1150 1160 |
| | 1200 1210 1220 1230 1240 |
| human | GGGGGGACATTGGAAAGGAGGGACCCCGCCACCCCAGCAGCTGAGGAGGG |
| | :: :: ::: :::::::: :: ::: :: |
| mouse | GG-----CAGTGGGAAGGAGGGGACCTCCCATGCC--------------- |
| | 1170 1180 1190 |
| | 1250 1260 1270 1280 1290 |
| human | GATTCTGGAACTGAATGGCGCTTCGGGATTCTGAGTAGCAGGGG-CAGCA |
| | :: :::: : :: :::::: ::: |
| mouse | -------CAA---------------GGATCCCCAGCGCCAGGGGACAG-- |
| | 1200 1210 |
| | 1300 1310 1320 1330 1340 |
| human | TGCCCAGTGGGCCTGGGGTCCCGGGAGGGATTCC--GGAATTGAGGGGCA |
| | ::::::: :::::::::::::::: ::::: ::: ::: ::: ::::: |
| mouse | TGCCCAGGGGGCCTGGGGTCCCGG-AGGGAGTCCTGGGATCTGAAGGGCA |
| | 1220 1230 1240 1250 1260 |
| | 1350 1360 1370 1380 |
| human | CGCAGGATTCTGAGCACC-------AGGGGCAGAGGCGGCC--AGACAAC |
| | : :::: ::::: :: ::: ::::::::::: : ::: |
| mouse | TTC--GATTGTGAGCGCCCAGGCAGAGGCGCAGAGGCGGCTGTACACAGG |
| | 1270 1280 1290 1300 1310 |
| | 1390 1400 1410 1420 1430 |
| human | CTCAGGGAGGAGTGTCCTGGCGTCCCCATCCTCCAAAGGGCCTGGGCCCG |
| | ::::: :::: : : :: :::: :::: |
| mouse | CTCAGAAAGGAAAGACTTGATGTCC---TCCT------------------ |
| | 1320 1330 1340 |
| | 1440 1450 1460 1470 1480 |
| human | CCCCGAGGGGGCAGCGAGAGGAGCTTCCCCATCC-CCGGTCAGTCCACCC |
| | : ::::::: :::::::: :: : :: :: :::: : |
| mouse | ------GAGGGCAGC-AGAGGAGC--GCCGAGCCGCCTGTCA-------C |
| | 1350 1360 1370 |
| | 1490 1500 1510 1520 1530 |
| human | TGCCCCGTCCACTTTCCCATCTCCTCGGTATAAATCATGTTTATAAGTTA |
| | : :::: ::::: :::: :: ::::::::::::::::::: |
| mouse | TTCCCCCTCCACC---------CCTCCATAGAAATCATGTTTATAAGTTA |
| | 1380 1390 1400 1410 |
| | 1540 1550 1560 1570 1580 |
| human | TGGAAGGACCGGGACAAAGCCGAATTCCAGCACACTGGCGGCCGTTACTA |
| | ::::: ::::::::: |
| mouse | TGGAAA-ACCGGGACAT--------------------------------- |
| | 1420 1430 |
| | 1590 1600 |
| human | GTGGATCCGAGCTCGGTACCAAG |
| | ----------------------- |

```
TGA = Stop codon.
The mouse p38.5 cDNA sequence was determined from cDNA clones primed with human primers EC4 and LP3, and extended with mouse primers EC4 and RD1 as described in Example 20.
Human Primers EC4: GCA GGC GAC CAT GGG GAA CGT (SEQ ID NO:3)
                            LP3: TGT CCC GGT TCT TCC ATA AC  (SEQ ID NO:4)
Mouse Primers EC4: GCA GGC GAC CAT GGG GAA CGT (SEQ ID NO:3)
                            RD1: ATG TCC CGG TTT TCC ATA AC  (SEQ ID NO:6)
The alignment was performed with the GeneStream Align program according to procedures described by Person, W. R., et al. (1997) Comparison of DNA sequences with protein sequences, Genomics 46: 24-36
Human p38.5 sequence 1609 nucleotides vs.
Mouse p38.5 sequence 1434 nucleotides.
Scoring matrix:, gap penalties: −12/−2
The human and mouse sequences show 75.5% nucleotide sequence identity and a Global alignment score of 3676.
```

TABLE 4

Alignment of the amino acid sequence of human p38.5 (SEQ ID NO:2)
with mouse p38.5 (SEQ ID NO:6)

| | |
|---|---|
| | 10 20 30 40 50 60 |
| Human | MGNVLAASSPPAGPPPPPAPALVGLPPPPPSPPGFTLPPLGGSLGAGTSTSRSSERTPGA |
| | ::::::::::::::::::::::::::::::::::::::::::.::.:.::.:.::::::: |
| Mouse | MGNVLAASSPPAGPPPPPTPSLVGLPPPPPSPPGFTLPPLGGGLGTGSSTGRGSERTPGA |
| | 10 20 30 40 50 60 |
| | 70 80 90 100 110 120 |
| Human | ATASASGAAEDGACGCLPNPGTFEECHRKCKELFPIQMEGVKLTVNKGLSNHFQVNHTVA |
| | :...:..:.:::.::::::::::::::::::::::.:::::::::::::::.:::.:::: |
| Mouse | AASGAAAASEDGSCGCLPNPGTFEECHRKCKELFPVQMEGVKLTVNKGLSNRFQVTHTVA |
| | 70 80 90 100 110 120 |
| | 130 140 150 160 170 180 |
| Human | LSTIGESNYHFGVTYVGTKQLSPTEAFPVLVGDMDNSGSLNAQVIHQLGPGLRSKMAIQT |
| | :.::: ::::::::::::::::::::::::::::::::::::::::::.::::::::::: |
| Mouse | LGTIGGSNYHFGVTYVGTKQLSPTEAFPVLVGDMDNSGSLNAQVIHQLSPGLRSKMAIQT |
| | 130 140 150 160 170 180 |
| | 190 200 210 220 230 240 |
| Human | QQSKFVNWQVDGEYRGSDFTAAVTLGNPDVLVGSGILVAHYLQSITPCLALGGELVYHRR |
| | :::::::::::::::::::::::::::::::::::::::::::::::::::::::::::: |
| Mouse | QQSKFVNWQVDGEYRGSDFTAAVTLGNPDVLVGSGILVAHYLQSITPCLALGGELVYHRR |
| | 190 200 210 220 230 240 |
| | 250 260 270 280 290 300 |
| Human | PGEEGTVMSLAGKYTLNNWLATVTLGQAGMHATYYHKASDQLQVGVEFEASTRMQDTSVS |
| | ::::::::::::::::::::::::::::::::::::::::::::::::::::::::::.: |
| | PGEEGTVMSLAGKYTLNNWLATVTLGQAGMHATYYHKASDQLQVGVEFEASTRMQDTSAS |
| Mouse | 250 260 270 280 290 300 |
| | 310 320 330 340 350 360 |
| Human | FGYQLDLPKANLLFKGSVDSNWIVGATLEKKLPPLPLTLALGAFLNHRKNKFQCGFGLTI |
| | :::::::::::.::::::.::::::::::::::::::::.: :::::::::: ::::::: |
| Mouse | FGYQLDLPKANFLFKGSVNSNWIVGATLEKKLPPLPLTLSLCAFLNHRKNKFLCGFGLTI |
| | 310 320 330 340 350 360 |
| Human | G |
| | : |
| Mouse | G |
| Human | p38.5 361 aa vs. |
| Mouse | p38.5 361 aa |

Scoring matrix:, gap penalties: −12/−2
92.8% sequence identity; Global alignment score: 2294
The bolded sequence from Phe185 to Arg195 in mouse and corresponds to the 11-mer peptide originally isolated from the purified native human protein. See Example 11. The 25-mer-peptide is shown in bold type at Ser43 to Gly67 of the human sequence. The mouse sequence differs in this region.

A recombinant plasmid, clone 32 (designated “nahNKCRp38.5”) comprising a fragment of human p38.5 cDNA was constructed using K562 cell RNA as template as described below in Example 20. This 730 bp fragment encodes an open reading frame that includes amino acids 101-324 of SEQ ID NO: 2. A microorganism deposit of nahNKCRp38.5 in compliance with the Budapest Treaty was made with the American Type Culture Collection (ATCC) located in Rockville, Md., on May 1, 1997, and assigned ATCC No. 98422.

A recombinant plasmid, clone 70 (designated “nahNKRp70”) containing a fragment of human p38.5 cDNA insert of 1.315 Kb (nucleotides 340-1635 of SEQ ID NO: 1) at the 3′ end of the gene was constructed as described in Example 20. A microorganism deposit of nahNKRp70 in compliance with the Budapest Treaty was made with the ATCC on May 1, 1997 and assigned ATCC No. 98424.

Also, a recombinant bacteriophage clone in the Lambda ZAP Express® vector (Stratagene, La Jolla, Calif.) containing cDNA encoding the entire naïve human NK cell library was constructed by subtractive hybridization with mRNA from human T-lymphocytes. The library was designated as “nahNKL” for non-activated human NK cell library. The library is particularly useful for isolating and characterizing genes and gene products that are unique to NK cells as compared to T cells. A deposit of the nahNKL library was made with the ATCC on May 1, 1997 in compliance with the Budapest Treaty and was assigned ATCC No. 98423.

These deposits were made under the provisions of the Budapest Treaty on the International Recognition of the Deposit of Microorganisms for the Purposes of Patent Procedure and the regulations thereunder (Budapest Treaty). This assures the maintenance of a viable culture for 30 years from date of deposit. The organisms will be made available by ATCC under the terms of the Budapest Treaty, and subject to an agreement between Applicants and ATCC which assures unrestricted availability upon issuance of the pertinent U.S. patent. Availability of the deposited strains is not to be construed as a license to practice the invention in contravention of the rights granted under the authority of any government in accordance with its patent laws.

Protein Methods

p38.5 may be isolated from mammals including, for example, humans and other primates, as well as pet animals such as dogs and cats, laboratory animals such as rats and mice, and farm animals such as horses, sheep, and cows. As used herein, the term “p38.5” includes the natural and recombinant proteins of 361 amino acids (SEQ ID NO: 2; shown in Table 2), as well as homologs and functional fragments thereof. Functional fragments of p38.5 may be any fragment that retains a functional activity of the 361 amino acid protein. The functional activity may be any functional activity of p38.5, such as for instance, antigenicity, immunogenicity or binding to naïve NK cells.

p38.5 may be prepared by any one of many methods that are known in the art. Such methods include isolating the protein directly from cells; isolating or synthesizing DNA encoding the protein and expressing the DNA to produce recombinant protein; and synthesizing the protein chemically from individual amino acids.

p38.5 may be isolated from cells, cell lysates, membranes or solubilized fractions or other cell components by standard methods. Some suitable methods include precipitation and liquid chromatographic protocols such as ion exchange, hydrophobic interaction and gel filtration. See, for example, Methods Enzymol (Guide to Protein Chemistry, Deutscher, ed., Section VII) pp. 182:309 (1990) and Scopes, Protein Purification, Springer-Verlag, New York (1987).

Alternatively, purified p38.5 may be obtained by separating the protein on preparative SDS-PAGE gels, slicing out the band of interest and electroeluting the protein from the polyacrylamide matrix by methods known in the art. The detergent SDS is removed from the protein by known methods, such as by dialysis or the use of a suitable column, such as the Extracti-Gel™ column from Pierce.

Moreover, p38.5 may be chemically synthesized by methods known in the art. Suitable methods for synthesizing proteins are described by Stuart and Young, Solid Phase Peptide Synthesis, 2d ed., Pierce Chemical Company (1984).

Further, p38.5 may also be prepared by providing DNA that encodes the protein; amplifying or cloning the DNA in a suitable host; expressing the DNA in a suitable host; and harvesting the protein.

Molecular weights are determined by resolving single bands by SDS-PAGE and comparing their positions to those of known standards according to the method of Laemmli, Nature 227:680-685 (1970). The method is accurate within a range of 3-5%. Molecular weights may vary slightly between determinations.

Determinations of whether two amino acid sequences are substantially homologous may, for the purpose of the present specification, be based on FASTA searches as taught by Pearson et al., Proc Natl Acad Sci USA 85:2444-2448 (1988). Substantially homologous p38.5 sequences have at least 90% identity, preferably at least 95% identity and, more preferably, at least 97% identity in amino acid sequence. Optimally, the homologous p38.5 sequences are at least 99% homologous.

As is also well known, it is possible to substitute amino acids in a sequence in a peptide or a protein such as the p38.5 with equivalent amino acids. Groups of amino acids known normally to be equivalent are:

(a) Ala (A), Ser (S), Thr (T), Pro (P), Gly (G);

(b) Asn (N), Asp (D), Glu (E), Gln (Q);

(c) His (H), Arg (R), Lys (K);

(d) Met (M), Leu (L), Ile (I), Val (V); and

(e) Phe (F), Tyr (Y), Trp (W).

Any substitutions, additions, and/or deletions in p38.5 may be made provided that the subtitution, addition or deletion mutant of p38.5 continues to satisfy the functional criteria, such as antigenic, immunological or NK cell-binding criteria described herein. An amino acid sequence that is substantially identical to another sequence, but that differs from another sequence by one or more substitutions, additions and/or deletions is considered to be an equivalent sequence. Preferably, less than 25%, more preferably less than 10%, of the number of amino acid residues in a sequence are substituted for, added to, or deleted from the fragments in the proteins of the invention.

It may be desirable that such structurally homologous proteins also exhibit functional homology, insofar as the homologous protein has substantially the same function as a protein of the invention. For example, structurally homologous proteins may be considered to be functionally homologous if they exhibit similar binding properties, or similar antigenicity or immunogenicity, etc.

However, two proteins or the nucleic acids encoding them may be considered to be substantially homologous in structure, and yet differ substantially in function. For example, single nucleotide polymorphisms (SNPs) among alleles may be expressed as proteins differing in only one amino acid, yet having substantial differences in antibody-binding or ligand-binding activity. Other structural differences, such as substitutions, deletions, splicing variants, and the like, may also affect the function of otherwise structurally identical or homologous proteins. Allelic variants of p38.5 are contemplated within the scope of the invention.

Fragments containing antigenic sequences may be selected on the basis of generally accepted criteria of predicted antigenicity. Such criteria include the hydrophilicity and relative antigenic index, as determined by surface exposure analysis of proteins. The determination of appropriate criteria is known to those skilled in the art, and has been described, for example, by Hopp et al., Proc Natl Acad Sci USA 78:3824-3828 (1981); Kyte et al., J Mol Biol 157:105-132 (1982); Emini, J Virol 55:836-839 (1985); Jameson et al., CA BIOS 4:181-186 (1988); and Karplus et al., Naturwissenschaften 72:212-213 (1985). Amino acid domains predicted by these criteria to be surface exposed are selected preferentially over domains predicted to be more hydrophobic or hidden from surface interactions. Methods for isolating and identifying antigenic fragments from known antigenic proteins are described by Salfeld et al., J Virol 63:798-808 (1989) and by Isola et al., J Virol 63:2325-2334 (1989).

p38.5 may be expressed in the form of a fusion protein with an appropriate fusion partner. The fusion partner preferably facilitates purification and identification. Increased yields may be achieved when the fusion partner is expressed naturally in the host cell. Examples of useful fusion partners include beta-galactosidase, Gray et al., Proc Natl Acad Sci USA 79:6598 (1982); trpE, Itakura et al., Science 198:1056 (1977); protein A, Uhlen et al., Gene 23:369 (1983); glutathione S-transferase, Johnson, Nature 338:585 (1989); maltose binding protein, Riggs, P., in Ausebel, F. M. et al (eds) Curr. Protocols in Mol. Biol., Greene Assocs/Wiley Interscience, NY (1990) and the cellulose binding domain (Shpigel E, et al. Biotechnol Bioeng (1999) 65(1):17-23).

Such p38.5 fusion proteins may be purified by affinity chromatography using reagents that bind to the fusion partner. The reagent may be a specific ligand of the fusion partner or an antibody, preferably a monoclonal antibody. For example, fusion proteins containing beta-galactosidase may be purified by affinity chromatography using an anti-beta-galactosidase antibody column. Ullman, Gene 29:27-31 (1984). Similarly, fusion proteins containing maltose binding protein may be purified by affinity chromatography using a column containing cross-linked amylose; see Guan, European Patent Application 286,239. The p38.5 may also be purified by means of a purification tag such as a polyhistidine tag. A polyhistidine sequence is added et either terminus or internally to the protein by recombinant methods and the protein is purified on a metal chelate column, such as a nickel affinity column.

p38.5 may be fused at the amino-terminal or the carboxy-terminal of the cleavage site. Optionally, the DNA that encodes the expressed fusion protein is engineered so that the fusion protein contains a cleavable site between p38.5 and the fusion partner. Both chemical and enzymatic cleavable sites are known in the art. Suitable examples of sites that are cleavable enzymatically include sites that are specifically recognized and cleaved by collagenase (Keil et al., FEBS Letters 56:292-296, 1975); enterokinase (Hopp et al., Bio/Tecnology 6:1204-1210 (1988)); factor Xa (Nagai et al., Methods Enzymol 153:461-481 (1987)); and thrombin (Eaton et al., Biochemistry 25:505 (1986)). Collagenase cleaves between proline and X in the sequence Pro-X-Gly-Pro wherein X is a neutral amino acid. Enterokinase cleaves after lysine in the sequence Asp-Asp-Asp-Asp-Lys. Factor Xa cleaves after arginine in the sequence Ile-Glu-Gly-Arg. Thrombin cleaves between arginine and glycine in the sequence Arg-Gly-Ser-Pro. Specific chemical cleavage agents are also known. For example, cyanogen bromide cleaves at methionine residues in proteins.

Recombinant p38.5 may be purified by methods well known in the art. Such methods include affinity chromatography using specific antibodies. Alternatively, the recombinant protein may be purified using a combination of ion-exchange, size-exclusion, and hydrophobic interaction chromatography. These and other suitable methods are described, for example, by Marston, “The purification of eukaryotic proteins expressed in E. coli,” in DNA Cloning, D M Glover, ed., Volume III, IRL Press Ltd., England (1987); Marston F A O and Hartley D L, “Solubilization of protein aggregates,” pp. 266-267 in Guide to Protein Purification, Deutscher M P, ed., Academic Press, San Diego (1990) and Sambrook J. and Russel D W. (2001) id.

Antibody Methods

The present invention further provides antibodies raised against p38.5. An “antibody” in accordance with the present specification is intended to encompass any protein that specifically binds an epitope. The antibody may be polyclonal or monoclonal. Antibodies further include recombinant single chain antibodies or monoclonal Fab fragments prepared in accordance with the method of Huse et al., Science 246:1275-1281 (1989).

Polyclonal antibodies are isolated from mammals that have been inoculated with p38.5 or a fragment or a functional analog thereof in accordance with methods that are well known in the art. Briefly, polyclonal antibodies may be produced by injecting a host mammal, such as a rabbit, mouse, rat, or goat, with the protein or a fragment thereof capable of producing antibodies that distinguish between mutant and wild-type protein. The peptide or peptide fragment injected may contain the wild-type sequence or the mutant sequence. Sera from the mammal are extracted and screened to obtain polyclonal antibodies that are specific to the peptide or peptide fragment.

The antibodies are preferably monoclonal. Monoclonal antibodies may be produced by methods known in the art. These methods include the immunological method described by Kohler and Milstein, Nature 256:495-497 (1975) and by Campbell, in Burdon et al., eds, Laboratory Techniques in Biochemistry and Molecular Biology, Vol. 13, Elsevier Science Publishers, Amsterdam (1985); as well as the recombinant DNA method described by Huse et al., Science 246:1275-1281 (1989).

To produce monoclonal antibodies, a host mammal is inoculated with a peptide or peptide fragment or homolog as described above, and then boosted with a second inoculation of the peptide or fragment. Spleens are collected from inoculated mammals a few days after the final boost. Cell suspensions from the spleens are fused with a tumor cell in accordance with the general method described by Kohler and Milstein (1975). See also Campbell (1985) Id. To be useful, a peptide fragment must contain sufficient amino acid residues to encompass an epitope of p38.5, the fragment or the homolog.

If the fragment is too short to be immunogenic, it may be conjugated to a carrier molecule. Suitable carrier molecules include keyhole limpet hemocyanin and bovine serum albumin. Conjugation may be carried out by methods known in the art. One such method is to combine a cysteine residue of the fragment, or a cysteine reisdue added to the N-terminus or the C-terminus, with a cysteine residue on the carrier molecule to form an S-S bond to the carrier molecule.

Useful methods for preparing polyclonal and monoclonal antibodies that exhibit specificity toward single amino acid differences between oncogenes are described by McCormick et al., U.S. Pat. No. 4,798,787 and are incorporated herein by reference.

Antibodies may be engineered using genetic techniques to produce chimeric antibodies including protein components from two or more species. For use in in vivo applications with human subject, the antibody may be humanized, containing an antigen binding region from, e.g., a rodent, with the bulk of the antibody replaced with sequences derived from human immunoglobulin. Methods are also known for inducing expression of engineered antibodies in various cell types, such as mammalian and microbial cell types. Numerous techniques for preparing engineered antibodies are described, e.g., in Owens R J and Young R J, “The genetic engineering of monoclonal antibodies,” J Immunol Meth 168:149-165 (1994).

Assays for directly detecting the presence of proteins such as p38.5 on or in cells with antibodies follow known formats, such as, fluorescence activated flow cytometry, fluorescence microscopy, and immunohistochemical-electron microscopy. Moreover, in general assays for detecting the presence of proteins using antibodies have been previously described and follow known formats, such as standard blot and ELISA formats. These formats are normally based on incubating an antibody with a sample to be tested for the protein and detecting the presence of a complex between the antibody and the protein. The antibody is labeled either before, during, or after the incubation step. The protein may be immobilized prior to detection. Immobilization may be accomplished by directly binding the protein to a solid surface, such as a microtiter well, or by binding the protein to immobilized antibodies.

Immunoassays may involve one step or two steps. In a one-step assay, the target molecule, if it is present, is immobilized and subsequently incubated with a labeled antibody. The labeled antibody binds to the immobilized target molecule. After washing to remove unbound molecules, the sample is assayed for the presence of the label.

In a two-step assay, immobilized target molecule is incubated with an unlabeled first antibody. The target molecule-antibody complex, if present, is then bound to a second, labeled antibody that is specific for the unlabeled antibody. The sample is washed and assayed for the presence of the label, as described above.

The immunometric assays described above include simultaneous sandwich, forward sandwich, and reverse sandwich immunoassays. These terms are well known to those skilled in the art.

In a forward sandwich immunoassay, a sample is first incubated with a solid phase immunoabsorbent containing antibody against the protein. Incubation is continued for a period of time sufficient to allow the protein in the sample to bind to the immobilized antibody in the solid phase. After the first incubation, the solid phase immunoabsorbent is separated from the incubation mixture and washed to remove excess protein and other interfering substances which also may be present in the sample. Solid phase immunoabsorbent-containing protein bound to the immobilized antibodies is subsequently incubated for a second time with soluble labeled antibody cross-reactive with a different epitope on the protein. After the second incubation, another wash is performed to remove the unbound labeled antibody from the solid immunoabsorbent and to remove non-specifically bound labeled antibody. Labeled antibody bound to the solid phase immunoabsorbent is then detected and the amount of labeled antibody detected serves as a direct measure of the amount of antigen present in the original sample. Alternatively, labeled antibody that is not associated with the immunoabsorbent complex may also be detected, in which case the measure is in inverse proportion to the amount of antigen present in the sample. Forward sandwich assays are described, for example, in U.S. Pat. Nos. 3,867,517 to Ling and 4,376,110 to David et al.

In a reverse sandwich assay, the sample is initially incubated with labeled antibody. The solid phase immunoabsorbent containing immobilized antibody cross- reactive with a different epitope on the protein the labeled antibody is added, and a second incubation is carried out. The initial washing step required by a forward sandwich assay is not required, although a wash is performed after the second incubation. Reverse sandwich assays are described, for example, in U.S. Pat. Nos. 4,098,876 to Piasio et al. and 4,376,110 to David et al.

In a simultaneous sandwich assay, the sample, the immunoabsorbent with immobilized antibody, and labeled soluble antibody specific to a different epitope are incubated simultaneously in one incubation step. The simultaneous assay requires only a single incubation and does not require any washing steps. The use of a simultaneous assay is a very useful technique, providing ease of handling, homogeneity, reproducibility, linearity of the assays, and high precision. See U.S. Pat. No. 4,376,110 to David et al.

There are many solid phase immunoabsorbents which have been employed and which may be used in the present invention. Well known immunoabsorbents include beads formed from glass, polystyrene, polypropylene, dextran, nylon, and other material; and tubes formed from or coated with such materials, and the like. The immobilized antibodies may be covalently or physically bound to the solid phase immunoabsorbent, by techniques such as covalent bonding via an amide or ester linkage or by absorption.

The p38.5 may be used to detect the presence of antibodies specific for the protein in a sample. In one embodiment, the p38.5 may be labeled and used as a probe in standard immunoassays to detect antibodies against the proteins in samples, such as in blood or other bodily fluids of patients being tested for the presence of NK-susceptible tumors or NK cells. In general, a protein in accordance with the invention is incubated with the sample suspected of containing antibodies to the protein. The protein is labeled either before, during, or after incubation. The detection of labeled protein bound to an antibody in the sample indicates the presence of the antibody. The antibody is preferably immobilized.

Suitable assays are known in the art, such as the standard ELISA protocol described by R H Kennett, “Enzyme-linked antibody assay with cells attached to polyvinyl chloride plates” in Kennett et al., Monoclonal Antibodies, Plenum Press, New York, pp. 376 et seq. (1981).

Briefly, plates are coated with antigenic protein at a concentration sufficient to bind detectable amounts of the antibody. After incubating the plates with the protein, the plates are blocked with a suitable blocking agent, such as, for example, 10% normal goat serum. The sample, such as patient sera, is added and titered to determine the endpoint. Positive and negative controls are added simultaneously to quantitate the amount of relevant antibody present in the unknown samples. Following incubation, the samples are probed with goat anti-human Ig conjugated to a suitable label, such as an enzyme. The presence of anti-p38.5 antibodies in the sample is indicated by the presence of the bound label.

For use in immunoassays, the probe may be the entire protein or may be a functional analog thereof. Functional analogs of these proteins include fragments and substitution, addition and deletion mutations that do not destroy the ability of the proteins to bind to their antibodies. As long as the proteins are able to detect antibodies specific for the protein, they are useful in the present invention.

Nucleic Acid Methods

“Nucleic acid,” as used herein, means any nucleic acid, including deoxyribonucleic acid (DNA) and ribonucleic acid (RNA). Nucleic acids useful for methods of the present invention may be a nucleic acid that encodes p38.5 or a fragment thereof.

Alternatively, the nucleic acid may be a primer suitable for priming from a nucleic acid template from a mammalian gene encoding p38.5, or the nucleic acid may be a probe suitable for hybridizing to nucleic acid target gene encoding p38.5 from a mammalian source. The fragment may be an oligonucleotide (i.e., from about 8 nucleotides to about 50 nucleotides in length) or a polynucleotide (from about 50 to about 2,000 or even 5,000 or more nucleotides in length). For example, nucleic acids include unspliced primary transcript RNA, messenger RNA (mRNA), complementary DNA (cDNA), genomic DNA, synthetic DNA or RNA, and the like. The nucleic acid may be single stranded, or partially or completely double stranded (duplex). Duplex nucleic acids may be homoduplex or heteroduplex.

A nucleic acid may be considered to be homologous to a nucleic acid encoding a p38.5 amino acid sequence if nucleic acids hybridize with one another. Highly homologous nucleic acids hybridize under stringent hybridization conditions. Stringent hybridization conditions include for instance hybridization at 65° C. in 6×SC (0.9M sodium chloride, 0.09M sodium citrate), 10 mM Tris-HCl, pH 7.0, 1 mM EDTA. The relationships between hybridization conditions and degree of homology are understood by those skilled in the art. See for example, Sambrook J, and Russel D W. (2001) Id.

The invention specifically includes nucleic acids that have a nucleotide sequence including the sequence defined by SEQ ID NO: 1, or a homolog thereof, or a unique fragment thereof. As used herein, the sequence of a nucleic acid molecule that encodes p38.5 is considered homologous to a second nucleic acid molecule if the nucleotide sequence of the first nucleic acid molecule is at least about 60% identical, preferably at least about 70% identical, and more preferably at least about 80% identical to the sequence of the second molecule. More preferably still, the molecules are at least 90% identical, yet more preferably at least 95% identical and optimally at least 98% identical.

p38.5-encoding nucleic acids and fragments include primers and probes which are useful as tools in many molecular engineering techniques. The fragments maybe used as primers (“amplimer”) to selectively amplify nucleic acid, such as genomic DNA, total RNA, or polyA RNA etc. The fragments may also be oligonucleotides complementary to a target nucleic acid molecule suitable for use as a probe.

The length of the oligonucleotide probe is not critical, provided it is capable of hybridizing to the target molecule. The oligonucleotide should contain at least 6 nucleotides, preferably at least 10 nucleotides, and more preferably at least 15 nucleotides. It is preferred that the probe hybridize to a sequence that is unique to the sequence encoding p38.5. Such probes are said to hybridize specifically with their target sequences. There is no upper limit to the length of the oligonucleotide probes. Longer probes are more difficult to prepare and require longer hybridization times. Therefore, the probe should not be longer than necessary. Normally, the oligonucleotide probe will not contain more than 50 nucleotides, preferably not more than 40 nucleotides, and more preferably not more than 30 nucleotides.

Oligonucleotide probes and primers may be provided as homogeneous preparations of identical oligonucleotides, or as heterogeneous preparations of non-identical and/or non-overlapping sets of oligonucleotides. For example, in amplification processes, pairs of primers may be used that are complementary to the portions of the two opposing anti-parallel strands of double stranded DNA.

Methods for making and using nucleic acid probes are well documented in the art. For example, see Keller G H and Manak M M, DNA Probes, 2d ed., Macmillan Publishers Ltd., England (1991) and Hames B D and Higgins S J, eds., Gene Probes I and Gene Probes II, IRL Press, Oxford (1995). See also Sambrook J. and Russel D. W. Eds. Molecular cloning: A Laboratory Manual 3d Ed. Cold Spring Harbor Laboratory Press, Cold Spring Harbor, N.Y. 2001.

The DNA may be synthesized chemically from the four nucleotides in whole or in part by methods known in the art. Such methods include those described by Caruthers M H, Science 230:281-285 (1985). DNA may also be synthesized by preparing overlapping double-stranded oligonucleotides, filling in the gaps, and ligating the ends together. See, generally, Sambrook J. and Russel D. W. (2001) Id. and Glover D M and Hames B D, eds., DNA Cloning, 2d ed., Vols. 1-4, IRL Press, Oxford (1995).

The DNA may be cloned in a suitable host cell and expressed. The DNA and protein may be recovered from the host cell. See, generally, Sambrook and Russel. (2001) Id., for methods relating to the manufacture and manipulation of nucleic acids.

Methods well known in the are may be used for isolating DNA encoding a particular protein such as p38.5, once the protein has been isolated and purified. Many of these methods are described in Sambrook and Russel (2001) Id. In this way the full nucleic acid sequence of the gene for the proteins of the invention may be obtained. The immunological screening method is a preferred method.

To illustrate the method, chromosomal DNA is isolated and cleaved into fragments of suitable size by standard methods. Suitable DNA cleavage methods include, for example, sonication, limited DNAse digestion and restriction endonuclease digestion. A suitable average fragment size is approximately 500 bp. Linkers are added to the fragments and the resulting fragments are digested with the cognate restriction enzyme and ligated to a suitable vector. The linker site is designed to correspond to a restriction site in the vector. Suitable linkers include, for example, linkers encoding a EcoRI, PstI or BamHI site. A suitable vector is lambda-gt11. Ligated DNA may be packaged by commercial kits, such as a kit manufactured by Promega. Proteins from the resulting library are expressed in a suitable host, typically E. coli. The plaques that are obtained are screened immunologically by methods known in the art. Sambrook and Russel. (2001) Id. Screening may be facilitated by the use of a commercial screening kit, such as the picoBlue Immunological Screening Kit of Stratagene (La Jolla, Calif.) in accordance with the manufacturer's instructions.

Plaques that bind the protein-specific antibody are selected from non-reacting plaques and purified. Sambrook and Russel (2001) Id. The DNA from purified phage is isolated by methods known in the art. Suitable methods include, for example, polyethylene glycol precipitation, phage lysis, and anion exchange chromatography, which may be facilitated by the use of a kit manufactured by Qiagen (Studio City, Calif.).

The limits of the coding sequence may be determined by methods known in the art, such as by insertional mutagenesis.

DNA encoding p38.5 of the invention from other mammalian sources may be isolated by using the amino acid sequence of the coding region SEQ ID NO: 2 provided above or a fragment thereof to prepare one or more oligonucleotide probes. The probe is labeled and used to screen a genomic or cDNA library from the desired mammalian source in a suitable vector, such as in phage lambda. The cDNA library may by prepared from mRNA by known methods, such as those described by Gubler and Hoffman, Gene 25:263-270 (1983). The DNA isolated is sequenced, and the sequence used to prepare additional oligonucleotide probes. This procedure may be repeated to obtain overlapping fragments until a complete open reading frame is produced.

Given the nucleic acid sequence disclosed herein, the artisan may further design nucleic acid structures having particular functions in various types of applications. For example, the artisan may construct oligonucleotides or polynucleotides for use as primers in nucleic acid amplification procedures, such as the polymerase chain reaction (PCR), ligase chain reaction (LCR), Repair Chain Reaction (RCR), PCR oligonucleotide ligation assay (PCR-OLA), and the like.

PCR is a particularly useful technique, which may be applied to the nucleic acid detection methods of the present invention. PCR permits rapid amplification of a nucleic acid having an intervening nucleic acid sequence flanked by known sequences. In PCR, the known sequence information is used to design single-stranded primers which will hybridize to the nucleic acid. After annealing, the primers are extended by thermostable polymerase. The extended products are then removed from the nucleic acid so that a new cycle of annealing and extension can be performed. Successive cycles of annealing and extension leads to amplification of the target nucleic acid. Primers are chosen so that the amplified product is of a size that is easily detectable. Amplified products of 50-500 bp are easliy detected on agarose or polyacrylamide gels and are preferred, with products optimally being 200-400 bp in length.

The accessibility of methods for small scale purification of total RNA allows real-time PCR analysis to be performed with samples isolated from single cells. See for example, Dolter K. and Braman J, BioTechniques 30(6): 1358-1361.

Oligonucleotides encoding partial p38.5 sequences may be constructed for use as probes in hybridization studies, such as in situ hybridization and modified with a detectable tag or a fluorescent label for use in in situ hybridzation (FISH). Numerous methods for detectably labeling such probes with radioisotopes, fluorescent tags, enzymes, binding moieties (e.g., biotin), and the like are known, so that the p38.5 probes may be adapted for ease of detection.

Oligonucleotides may also be designed and manufactured for other purposes. For example, the invention enables the artisan to design antisense oligonucleotides, and triplex-forming oligonucleotides, and the like, for use in the study of structure/function relationships. Homologous recombination may be implemented by adaptation of the nucleic acid of the invention for use as targeting means.

Nucleic acids encoding p38.5 or fragments thereof may further be modified to alter biophysical or biological properties by means of techniques known in the art. For example, the nucleic acid may be modified to increase its stability against nucleases (e.g., “end-capping”), or to modify its lipophilicity, solubility, or binding affinity to complementary sequences. Methods for modifying nucleic acids to achieve specific purposes are disclosed in the art, for example, in Sambrook and Russel (2001) Id., the disclosure of which is incorporated by reference herein. Moreover, the nucleic acid may include one or more portions of nucleotide sequence that are non-coding for the p38.5.

The skilled artisan will appreciate that, if an amino acid sequence (primary structure) is known, a family of nucleic acids may then be constructed, each having a sequence that differs by at least one nucleotide, but where each different nucleic acid nevertheless encodes the same protein. For example, if a protein has been sequenced but its corresponding gene has not been identified, the gene may be acquired through amplification of genomic DNA using a set of degenerate primers that specify all possible sequences encoding the protein.

The entire gene or additional fragments of the gene are preferably isolated by using the known DNA sequence or a fragment thereof as a probe. To do so, restriction fragments from a genomic or cDNA library are identified by Southern hybridization using labeled oligonucleotide probes derived from SEQ ID NO: 1.

The DNA obtained may be amplified by methods known in the art. One suitable method is the polymerase chain reaction (PCR) method described by Saiki et al., Science 239:487 (1988), Mullis et al in U.S. Pat. No. 4,683,195 and by Sambrook and Russel. id (2001). It is convenient to amplify the clones in the lambda-gt10 or lambda-gt11 vectors using lambda-gt10 or lambda-gt11-specific oligomers as the amplimers (available from Clontech, Palo Alto, Calif.).

DNA encoding functional homologs of p38.5 may be prepared from wild-type DNA by site-directed mutagenesis. See, for example, Zoller M J, and Smith M, Nucleic Acids Res 10:6487-6500 (1982); Zoller M J, Methods Enzymol 100:468-500 (1983); Zoller M J, DNA 3(6):479-488 (1984); and McPherson M J, ed., Directed Mutagenesis. A Practical Approach, IRL Press, Oxford (1991); Sambrook and Russel (2001) Id.

Labeled Probes

The nucleic acid or protein (including antibody) probes described herein may be detectably labeled in accordance with methods known in the art. In general, the probe may be modified by attachment of a detectable label moiety to the probe, or a detectable probe may be manufactured with a detectable label moiety incorporated therein. The detectable label moiety may be any detectable moiety, many of which are known in the art, including radioactive atoms, electron dense atoms, enzymes, chromogens and colored compounds, fluorogens and fluorescent compounds, members of specific binding pairs, and the like.

Methods for labeling oligonucleotide probes have been described, for example, by Leary et al., Proc Natl Acad Sci USA (1983) 80:4045; Renz and Kurz, Nucl Acids Res 12:3435 (1984); Richardson and Gumport, Nucl Acids Res 11:6167 (1983); Smith et al., Nucl Acids Res 13:2399 (1985); Meinkoth and Wahl, Anal Biochem 138:267 (1984). Other methods for labeling nucleic acids are described, for example, in U.S. Pat. Nos. 4,711,955, 4,687,732, 5,241,060, 5,244,787, 5,328,824, 5,580,990, and 5,714,327, each of which is incorporated herein by reference.

Methods for labeling antibodies have been described, for example, by Hunter et al. (1962) and by David et al., Biochemistry 13:1014-1021 (1974). Additional methods for labeling antibodies are described in U.S. Pat. Nos. 3,940,475 to Gross.

The label moiety may be radioactive. Some examples of useful radioactive labels include ³²P, ¹²⁵I, ¹³¹I, and ³H. Use of radioactive labels have been described in U.K. patent document No. 2,034,323, U.S. Pat. Nos. 4,358,535 to Falkow et al. and 4,302,204 to Wahl et al., each incorporated herein by reference.

Some examples of non-radioactive labels include enzymes, chromogens and chromophores, atoms and molecules detectable by electron microscopy, and metal ions detectable by their magnetic properties.

Some useful enzymatic labels include enzymes that cause a detectable change in a substrate. Some useful enzymes (and their substrates) include, for example, horseradish peroxidase (pyrogallol and o-phenylenediamine), beta-galactosidase (fluorescein beta-D-galactopyranoside), luciferase (luciferin) and alkaline phosphatase (5-bromo-4-chloro-3-indolyl phosphate/nitro blue tetrazolium). The use of enzymatic labels has been described, for example, in U.K. 2,019,404, EP 63,879.

Useful reporter moieties include, for example, fluorescent, phosphorescent, chemiluminescent, and bioluminescent molecules, as well as dyes. Alternatively, the reoprter moiety may be green fluorescent protein (GFP), which is preferably part of a p38.5 fusion protein. Some specific colored or fluorescent compounds useful in the present invention include, for example, fluoresceins, coumarins, rhodamines, Texas red, phycoerythrins, umbelliferones, LUMINOL®, and the like. Chromogens or fluorogens, i.e., molecules that may be modified (e.g., oxidized) to become colored or fluorescent or to change their color or emission spectra, are also capable of being incorporated into probes to act as reporter moieties under particular conditions.

The label moieties may be conjugated to the probe by methods that are well known in the art. The label moieties may be directly attached through a functional group on the probe. The probe either contains or may be caused to contain such a functional group. Some examples of suitable functional groups include, for example, amino, carboxyl, sulfhydryl, maleimide, isocyanate, and isothiocyanate.

Alternatively, label moieties such as enzymes and chromogens may be conjugated to antibodies or nucleotides by means of coupling agents, such as dialdehydes, carbodiimides, dimaleimides, and the like.

The label moiety may also be conjugated to the probe by means of a ligand attached to the probe by a method described above and a receptor for that ligand attached to the label moiety. Any of the known ligand-receptor binding pair combinations is suitable. Some suitable ligand-receptor pairs include, for example, biotin-avidin or biotin-streptavidin, and antibody-antigen. The biotin-avidin combination is preferred.

Methods of Use of p38.5, probes for p38.5 and nucleic acids encoding p38.5

In accordance with the present invention, diagnostic reagents and therapeutic agents may be derived from the above-described genes or gene products. One preferred diagnostic method includes the use of an antibody or fragment thereof that has specific binding affinity for one of the expressed gene products p38.5, or characteristic fragments thereof. One such antibody is an antibody raised against the internal 11 mer peptide found within p38.5: PheValAsnTrpGlnValAspGlyGluTyrArg (SEQ ID NO: 7).

Therapeutic agents include, for example, cytotoxic agents bound to p38.5, or fragments thereof. Other therapeutic agents include antibodies or fragments raised against p38.5, or fragments thereof. Antibodies may be bound to cytotoxic agents for targeted delivery of the cytotoxic agents to NK-susceptible tumors or NK cells. In fact, p38.5 alone may be used for immunosuppression since this protein significantly reduces NK cell activity as demonstrated herein.

In accordance with the present invention, the diagnostic reagents and methods and the therapeutic agents may be used to detect and treat, i.e. inhibit the growth of, a variety of malignancies, in addition to other conditions involving NK cell-specific receptor-ligand interactions. Examples of NK-susceptible cancers suitable for detection and treatment through the use of the present invention include, but are not limited to, leukemias and lymphomas. NK-resistant cancers may also be treated if surface expression of p38.5 is increased thereby rendering the cancers NK susceptible. Other conditions involving NK cell-specific receptor-ligand interactions include organ and tissue transplantation (including bone marrow), infectious diseases and autoimmune diseases.

The proteins and functional analogs of the invention may also be used to produce antibodies for use as probes to detect the presence of p38.5 in a sample. The antibodies may be polyclonal or monoclonal. For this purpose, functional analogs include fragments and substitution, addition and deletion mutations of the protein as long as the analogs also produce antibodies capable of detecting the presence of p38.5 in a sample. The sample may, for example, be a cell sample, a biological fluid such as a bodily fluid or a cell lysate or cell membrane fraction from a mammal, including a human, to be tested for a tumor that expresses p38.5. The tumor may or may not be a naïve NK-susceptible tumor. The antibodies may be used as components of a kit for detection, diagnosis or monitoring of tumors expressing p38.5; the kits include an anti-p38.5 antibody and a second antibody that specifically binds the anti-p38.5 antibody.

The detection may be in vivo or in vitro. The in vivo assays are capable of detecting cell surface located p38.5, for example for imaging purposes or for targeted delivery of toxins or drug molecules. The in vitro assays may be performed with lysed or intact cells and are therefore capable of detecting intracellular p38.5 as well as cell surface located p38.5.

The invention further includes a method of treating cancers or immune reaction conditions that involve the protein-protein interaction described herein. For example, a mutant form of either p38.5 (or a fragment thereof) may be used to modulate, for example by inactivating or interfering with or even enhancing the function such as naïve NK cell binding, normally associated with binding of these proteins. The methods of treatment may be in vivo or ex-vivo. In ex-vivo treatment methods, the cell sample is obtained from the subject and is treated outside the body. After treatment the cell sample may be returned to the subject, as in for example methods of removal of cells with surface located p38.5 by passing the blood over a column with immobilized anti-p38.5 antibody. The treated blood is then returned to the subject in a continuous process.

In still another embodiment, the invention includes a method for killing tumor cells, comprising administering to an individual having a cytotoxic agent bound, attached or conjugated to an antibody that specifically binds p38.5. In this embodiment, the cytotoxic agent may be selected from radioactive agents such as ¹³¹I, protein toxins such as ricin A chain, alkylating agents such as chlorambucil and doxorubicin, antibiotics, e.g., DNA-binding antibiotics such as daunomycin, heavy metal complexes such as cisplatin, and anti-metabolites such as 5-fluorouracil and vinca alkaloids.

Autoimmune conditions amenable to treatment according to the method include, for example, allograft rejection, xenograft rejection, and graft versus host disease.

In the present study several strategies were employed to over come the difficulties of heterogeneity and the low abundance of NK cells in HPBL preparations. Only freshly isolated (i.e., non-cultured) NK cells were utilized.

Cells from individuals that demonstrated activity against NK resistant-LAK sensitive tumor cell lines were not utilized since the activated NK cells in these fresh preparations might mask the detection of naïve NK cell specific ligands. Finally the tagged-ligand cell adsorption technique of Das et al., J Exp. Med. 180: 273-281 (1994), was utilized with enriched NK cell preparations to increase the likelihood of detection of tumor proteins that selectively bound to the naïve NK cells.

Using the above approaches and additional biochemical and immunologic techniques we demonstrate for the first time a novel p38.5 on the plasma membrane of certain tumor cell lines that preferentially reacts with a surface component of naïve human NK cells. The interaction appears to be unique to NK cells since T lymphocytes did not bind p38.5. Binding studies have not, however been conducted with B cells, monocytes or polymorphonuclear leukocytes.

Additional evidence of a role for p38.5 as a target ligand in naïve NK cell mediated cytotoxicity is provided by data demonstrating an association of the expression of this molecule and susceptibility to cytolysis of different tumor cell lines. Flow cytometry and immunoprecipitation studies (of surface labeled cells) revealed that p38.5 is expressed on NK susceptible targets such as K562, MOLT-4 and Jurkat, whereas this molecule was not detected on the plasma membrane of NK resistant, LAK sensitive, targets such as Raji, A549 and MDA-MB-231 suggesting that p38.5 is not involved in LAK mediated cytotoxicity.

The functional role of p38.5 in NK cell mediated cytolysis was also demonstrated in studies of p38.5 loss variants. Following long term culture of wild type NK sensitive Jurkat and Molt-4 cell lines, variants were isolated that exhibited decreased levels of p38.5 and reduced susceptibility to lysis by NK cells. This property was not due to a phenotypic alteration in the cells as a result of culture conditions since resistant clones were obtained at limiting dilution. These studies clearly establish a strong association between the expression of p38.5 on the tumor plasma membrane and susceptibility to NK cell mediated cytolysis.

The invention provides methods of monitoring a disorder, disease or condition associated with a higher than normal level of expression of p38.5 in a subject herein illustrated by the examples below, the method comprises: providing two or more cell samples taken at different times from a subject to be tested; and

(a) determining the level of cell surface p38.5 in each of the cell samples, and comparing the level of cell surface p38.5 in each of the cell samples; or

(b) determining the level of total p38.5 in each of the cell samples, and comparing the level of total p38.5 in each of the cell samples; thereby monitoring the disease, disorder or condition.

The level of cell surface p38.5 may be determined by enzyme-linked immunosorbent assay (ELISA), radioimmunoassay (RIA), scintillation proximity (SPA), immunoprecipitation, Immunofluorescence, immunoelectrophoresis, immunodiffusion or agglutination assays.

The invention further provides methods of monitoring a disorder, disease or condition associated with a higher than normal level of expression of p38.5-specific RNA in a subject, the method comprising: providing two or more cell samples taken at different times from a subject to be tested, determining the level p38.5-specific RNA in each of the cell samples, and comparing the level p38.5-specific RNA in each of the cell samples, thereby monitoring the disease, disorder or condition.

The cell samples to be assayed may be obtained by surgical procedures, e.g., biopsy. The excised tissue may be assayed for the presence of an epitope of p38.5 which is recognized by an anti-p38.5 antibody as described above, e.g., the anti-11mer antibody, by methods generally known in the art, e.g., immunohistochemistry. The cell sample may be fixed or frozen to permit histological sectioning, and may be stained prior to incubation with the antibody. The antibody may be labeled, for example with a dye or fluorescent label, chemical, heavy metal or radioactive marker to permit the detection and localization of the antibody in the assayed tissue. Alternatively, other immunological assays known to those of skill in the art may be employed which permit detection of antibody binding to the excised tissue.

Body fluids such as blood and fluid blood components and peritoneal fluid, tissue and the like may be assayed for the presence and amount of p38.5. The assay may be performed by methods routinely used by those of skill in the art. Generally, a sample of body fluid is added to an assay mixture containing the antibody and a marker system for detection of antigen-bound antibody. Examples of such immunoassay systems are radioimmunoassays (RIA), enzyme-linked immunoassays (ELISA), immobilized immunoassays, and the like.

These methods are useful for monitoring the course or progress due to treatment of a disorder, disease or condition associated with a higher than normal level of expression of p38.5-specific RNA or of the p38.5 protein in a subject. The subject may also be undergoing treatment with other therapeutic agents, such as chemotherapeutic agents. Chemotherapeutic agents are understood to be exogenous substances suited and used to damage or destroy tumor cells. Here, in particular, cytostatic agents or derivatives thereof from the following group of cytostatic agents may be mentioned: alkylating agents such as, e.g., cyclophosphamide, chlorambucil, melphalan, busulfan, N-mustard compounds, mustargen; metal complex cytostatic agents such as metal complexes of platinum, palladium or ruthenium; antimetabolites such as methotrexate, 5-fluorouracil, cytorabin; natural substances such as vinblastine, vincristine, vindesine, etc.; antibiotic agents such as dactinomycin, daunorubicin, doxorubicin, bleomycin, mitomycin, etc.; hormones and hormone antagonists such as diethylstilbestrol, testolactone, tamoxifen, aminoglutethimide, and other compounds such as, e.g., hydroxyurea or procarbacin, as well as corticoids such as prednisolone, with cyclophosphamide being particularly preferred.

The invention further provides methods of treating a subject suffering from a tumor associated with a higher than normal level of expression of cell surface p38.5, comprising administering an effective amount of an antibody that specifically binds p38.5. The antibody may be a component of an immunoconjugate, comprising a toxin or a therapeutic agent.

The invention further provides methods for identifying a compound as an inhibitor of natural killer (NK) cell mediated killing of cells that express a higher than normal level of cell surface p38.5, comprising:

(i) providing a first sample of cells that expresses a higher than normal level of cell surface p38.5;

(ii) contacting the first sample of cells with the test compound;

(iii) contacting the first sample of cells with naïve NK cells, and

(iv) assessing the NK cell mediated cytotoxicity in the first sample of cells;

(v) providing a second sample of cells that expresses a higher than normal level of cell surface p38.5, identical to the first sample of cells;

(vi) contacting the second sample of cells with naïve NK cells, and

(vii) assessing the NK cell mediated cytotoxicity in the second sample of cells;

(viii) comparing the NK cell mediated cytotoxicity in the first sample of cells with the NK cell mediated cytotoxicity in the second sample of cells;

wherein a lower NK cell mediated cytotoxicity assessed in the first sample of cells than assessed in the first sample of cells identifies the compound as an inhibitor of natural killer (NK) cell mediated killing of cells that express a higher than normal level of cell surface p38.5. The compound identified may inhibit the natural killer (NK) cell mediated killing by inhibiting binding of p38.5 to NK cells, or may inhibit some step downstream from the binding of p38.5 to the NK cells. Preincubation with the test compound may also identify those compounds that inhibit expression of p38.5 in a form accessible to the NK cells. For example the compound may inhibit transcription or translation of the p38.5 gene. Alternatively, the compound may prevent proper folding or surface localization of the p38.5.

Cytotoxicity testing methods are well known in the art. See for example, U.S. Pat. No. 6,132,979 to Murakami, the entire specification of which is hereby incorporated by reference.

The invention also provides a method for determining the number of natural killer (NK) cells in a biological sample, comprising:

(a) contacting the sample containing NK cells with an antibody comprising a detectable label moiety and having specific binding affinity for p70 protein, under conditions permissive for binding of the antibody to p70 protein expressed by NK cells in said sample; and

(b) counting the number of cells bound by said antibody, thereby permitting determination the number of natural killer cells in the sample.

The following examples are provided to assist in a further understanding of the invention. The particular materials and conditions employed are intended to be further illustrative of the invention and are not limiting upon the reasonable scope thereof.

### EXAMPLES

### Chemicals, Antibodies and Cell Lines

N-hydroxy succinamide ester of biotin (biotin-NHS) was purchased from Calbiochem-Novabiochem Corp. (La Jolla, Calif.). Streptavidin alkaline phosphatase and all cell culture media and reagents were from Gibco BRL (Gaithersburg, Md.). Electrophoresis reagents and chemicals were purchased from Bio-Rad Laboratories (Melville, N.Y.).

RT-Titan one tube RT-PCR system kit and other molecular biology reagents were purchased from Roche (Indianapolis, Ind.). High fidelity RT-PCR kit was purchased from Epicenter Technologies (Madison, Wis.).

All other chemicals used were from Sigma Chemical Co. (St. Louis, Mo.).

Mouse anti-rabbit IgG was purchased from Pierce (Rockford, Ill.), goat anti-rabbit alkaline phosphatase was obtained from Sigma Chemical Co. (St. Louis, Mo.). Goat anti-rabbit IgG conjugated to FITC was obtained from Tago Inc. (Burlingame, Calif.).

Cell lines used in this study were obtained from the American Type Culture Collection (Rockville, Md.) and were maintained in media according to instructions provided. HUV-EC-C and CCD-27SK were grown with growth factors in specific endothelial cell culture and fibroblast cell culture media respectively purchased from Clonetics (Walkersville, Md.).

### Example 1

Preparation of Lymphocytes

Human peripheral blood lymphocytes (HPBL) were isolated as described in Das et al., J Exp. Med. 180: 273-281 (1994), which is herein incorporated by reference. In brief, peripheral blood was obtained from healthy volunteers, diluted 1:1 with HBSS, layered on Ficoll-Hapaque (Pharmacia, Piscataway, N.J.) and centrifuged at 200 g for 20 min. A mononuclear leukocyte fraction (Ficoll-Hypaque interface) was passed through nylon wool column twice to deplete B cells and macrophages. These cells were further processed to deplete T-cells and any remaining B-cells and macrophages.

First the cells were treated with Dynabead M-450 HLA class II antibody (Dynal Inc., Lake Success, N.Y.) and the antibody-absorbed cells were separated in a magnetic field. HLA class II nonreactive lymphocytes were successively treated with anti-CD3 and anti-CD5 (Pharmingen, San Diego, Calif.) and CD3 and CD5 reactive cells were separated by reacting the cell suspension with sheep anti-mouse IgG coated magnetic beads, as described in detail previously (Das et al., 1997). The resultant nonreactive lymphocyte populations were highly enriched in CD16 (85%) and mostly depleted of B-cells (<1%), macrophages (<1%) and T-cells (<3%). These cells were used as a source of mRNA for cDNA library construction and for analytical studies as described below. T-lymphocytes were positively selected using anti-CD3 magnetic beads as described previously (Das et al., 1997) and their mRNA was used for analytical studies.

### Example 2

Isolation of Naïve NK Cells and T Lymphocytes

Naïve human NK cells are defined as freshly isolated CD3⁻, CD5⁻, CD16⁺ that exhibit limited target cell specificity, i.e., lytic activity against K562 but not A549 tumor cells. Activated NK cells, i.e., those which are stimulated with lymphokines such as IL-2 (lymphokine activate killer cells, LAK), are able to lyse NK resistant tumor cells such as A549. See Trinchieri (1989) and Whiteside (1996). Only those preparations of HPBL that lacked LAK cell activity were used for the isolation of naïve NK cells and T lymphocytes. Isolation of naïve NK cells and T lymphocytes was performed as follows. Freshly isolated HPBL were suspended in phosphate buffered saline (PBS) containing 2% fetal bovine serum (FBS) at 20×10⁶ cells/ml and incubated (4° C. for 30 min) with anti-CD5, 20 μl/1×10⁶ cells (Becton-Dickinson, San Jose, Calif.).

Anti-CD5 reactive lymphocytes were captured by incubating the mixture with sheep anti-mouse IgG-coated magnetic beads, 30 μl/1×10⁶ cells (Dynal Inc., Lake Success, N.Y.). The magnetic beads containing adherent CD5⁺ cells were washed, suspended in RPMI supplemented with 15% FBS and incubated overnight at 37° C. in 5% CO₂. Subsequently CD-enriched lymphocytes (T lymphocytes) were separated from the magnetic beads by vortexing for 2 minutes and placing the mixture in a magnetic field. The supernatant containing CD5⁻HPBL was incubated with anti-CD3, 20 μl/1×10⁶ cells (Becton Dickinson, San Jose, Calif.) at 4° C. for 30 minutes. Anti-CD3-reactive lymphocytes were separated using magnetic beads as described above. The supernatant was centrifuged to obtain cells that were depleted of CD3⁺ and CD5⁺ lymphocytes (i.e., CD 16⁺ enriched naïve NK cells). The average phenotype of the T lymphocyte enriched fraction was <3% CD16⁺, 50% CD4⁺ and 93% CD3⁺. These cells did not exhibit NK or LAK cytolytic activity. The average phenotype of the negatively selected naïve NK cell enriched fraction was 85% CD16⁺, <3% CD3⁺. These cells exhibited increased NK cell lytic activity (60% cytotoxicity at an effector to target E:T ratios of 100:1) compared to unfractionated HPBL (32% cytotoxicity).

### Example 3

Binding of Tumor Plasma Membrane Proteins to Lymphocytes

Plasma membrane proteins of viable tumor cells were labeled with biotin as described Das et al. (1994). Briefly, cells (>99% viable by trypan blue dye exclusion) were washed three times with a solution containing 10 mM HEPES, 145 mM NaCl, 4 mM KCl, 11 mM glucose, pH 8.0 (buffer A) and then incubated in the same buffer with biotin-NHS (1 mM biotin-NHS/10×10⁶ cells) for one hour at 4° C. Cells were then washed three times with 20 vol of buffer A to remove unreacted biotin. The biotinylated cells were suspended in 250 mM sucrose with 10 mM HEPES (pH 8.0) and disrupted by N₂ cavitation (1000 psi) in a Parr's chamber. The resulting suspension was centrifuged at 200 g for 5 minutes to remove undisrupted cells and the supernatant was then centrifuged at 30,000 g for 20 minutes to obtain a crude membrane fraction. Membranes were washed once with 10 mM sodium borate, 10 mM benzamidine, 1 mM ethylenediamine tetraacetic acid (EDTA), 1 mM iodoacetamide, 1 mM phenyl methyl sulfonyl fluoride (PMSF), pH 8.0 (buffer B) and incubated in the same solution with 1% Triton X-100 at 4° C. overnight. Solubilized membrane proteins were obtained as the 30,000 g supernatant of the detergent treated membranes and dialyzed extensively against buffer B containing 0.05% Triton X-100, as described in Das et al. (1994).

Biotin labeled solubilized tumor plasma membrane proteins were reacted with freshly isolated naïve NK cells and T lymphocytes at a ratio of 2:1 (on the basis of cell numbers) in RPMI-1640 supplemented with 15% FBS at 4° C. for 2 h. The reacted cells were washed twice with media and three times with buffer A. Finally, cells were solubilized in Laemmli sample buffer and the solubilized proteins were subjected to SDS-PAGE and Western blotting. Biotinylated tumor membrane proteins that bound to lymphocytes were identified with a streptavidin biotin detection system as described in Das et al. (1994).

### Example 4

Purification of Tumor Membrane Proteins

Membrane proteins were purified as described Das et al. (1994). Approximately 1×10¹¹ cells of the erythroleukemia cell line (K562) were washed three times with PBS and used to prepare plasma membrane proteins as described above. Solubilized membrane proteins were extensively dialyzed against buffer B and subjected to preparative SDS-PAGE. A vertical portion of the gel was sliced and stained with Coomassie blue to locate desired proteins. Protein bands of interest were cut from the gel, eluted and further purified by SDS-PAGE.

A portion of the purified protein was extensively dialyzed against 10 mM Tris-HCl, pH 8.0 and used for cytotoxicity inhibition studies or dialyzed against Buffer B and labeled with biotin as described above for study of its binding properties to various lymphocyte subsets.

Another portion of the purified protein was also isolated on nitrocellulose paper by SDS-PAGE and transblotted using the method described by Aebersold et al. Proc. Natl. Acad. Sci 84: 6970-6974 (1987) for the purpose of internal amino acid sequence analysis of the protein subsequent to in situ protease digestion (Harvard Micro Chemistry Laboratory, Cambridge, Mass.). Aebersold et al. (1987) is herein incorporated by reference.

More generally the preparation of tumor or NK cell Plasma Membrane Proteins was carried out as follows. Tumor cells (>99% viable by trypan blue dye exclusion) were washed three times with a solution containing 10 mM HEPES, 145 mM NaCl, 4 mM KCl, 11 mM glucose, pH 8.0 (buffer A). Cells were suspended in 250 mM sucrose with 10 mM HEPES (pH 8.0) and disrupted by N₂ cavitation (1000 psi) in a Parr's chamber for 45 minutes. The resulting suspension was centrifuged at 200 g for 5 minutes to remove undisrupted cells and the supernatant was then centrifuged at 30,000 g for 20 minutes to obtain a crude membrane fraction. Membranes were washed once with 10 mM sodium borate, 10 mM benzamidine, 1 mM ethylenediamine tetraacetic acid (EDTA), 1 mM iodoacetamide, 1 mM phenyl methyl sulfonyl fluoride (PMSF), pH 8.0 (buffer B) and incubated in the same solution with 1% Triton X-100 at 4° C. overnight. Solubilized membrane proteins were obtained as the 30,000 g supernatant of the detergent treated membranes and dialyzed extensively against buffer B containing 0.05% Triton X-100. This procedure resulted in proteins from the membrane that were relatively water soluble.

Tumor membrane proteins were than purified as follows. Solubilized membrane proteins, as prepared above were incubated with Laemmli's sample buffer at 37° C. for 15 minutes and then subjected to separation based on their molecular weight by preparative SDS-PAGE. A vertical portion of the gel was sliced and stained with Coomassie blue to locate desired proteins. The p38.5 band was cut from the gel, eluted and further purified by SDS-PAGE in a similar manner. Finally the p38.5 band was eluted and dialyzed against a buffer of choice. The purity of the protein was established by subjecting the eluted protein to gel electrofocusing. Purified p338.5 was thus obtained by electroelution of the band from the second SDS-PAGE or from electrofocusing gel.

Purified p38.5, after extensively dialysis against an appropriate physiologic buffer, may be used for production of polyclonal antibodies and/or monoclonal antibodies. The p38.5 was used to alter the function of NK cells by direct binding to NK cells.

### Example 5

Immunoprecipitation of Surface-labeled Proteins

Immunoprecipitation of plasma membrane proteins was performed by initially incubating anti-p38.5 with anti-rabbit IgG coupled to CNBr-Sepharose 4B at 4° C. for 90 min in PBS containing 1% BSA. See Das et al. (1994). The reacted beads were then washed three times with buffer and incubated at 4° C. for 90 minutes with surface biotinylated tumor plasma membrane proteins. Beads were then washed three times with PBS containing 1% BSA and two times with PBS alone, suspended in Laemmli SDS-PAGE sample buffer and boiled for 10 minutes. The supernatant was subjected to SDS-PAGE and proteins transblotted to Immobilon-P membrane (Millipore Corp., Bedford, Mass.). Anti-p38.5 immunoprecipitated biotinylated tumor membrane proteins were identified on Western blots by reaction with a streptavidin-biotin detection system.

### Example 6

Fluorescence Activated Flow Cytometry

Approximately, 1×10⁶ cells were washed three times with PBS containing 0.1% BSA (PBS-BSA) and incubated with appropriately diluted anti-p38.5 at 4° C. for 30 minutes. Cells were then washed three times with PBS-BSA and reacted with FITC-F(ab′)₂ goat anti-rabbit IgG for 30 minutes at 4° C. Finally, cells were washed three times with PBS-BSA, resuspended to a concentration of 1×10⁶/ml and analyzed on a FACSort® Flow Cytometer (Becton Dickinson, San Jose, Calif.). See, for example, Current Protocols in Immunology, Coligan et al., eds., John Wiley & Sons (1991), which is herein incorporated by reference.

### Example 7

Lymphocyte Mediated Cytotoxicity

Cytolysis of tumor cells by lymphocytes was measured as described in Das et al. (1994) and Karpel and Norin, Chest 96:794-798 (1989). Briefly, 100 μl of ⁵¹Cr-labeled target cells (2×10⁴ cells/ml) was mixed with 100 μl of lymphocytes (2×10⁶ cells/ml) to yield serial two fold dilutions of E/T cell ratios of 100:1 to 6:1. Cells were incubated at 37° C. for 3 hours in 5% CO₂. Incubation of target cells without the effector cells and in media alone served as a control for the spontaneous release of ⁵¹Cr, and wells without effector cells but with 1% SDS provided the maximum amount of radioactivity present. Cytolysis was calculated as percent Cytotoxicity=100×[(CPMₑₓₚ−CPMₛₚₒₙₜ)/(CPMₘₐₓ−CPMspont.)].

### Example 8

Binding of Tumor Plasma Membrane Protein p38.5 to Human Lymphocytes

A tagged ligand-cell adsorption technique, see Das et al. (1994), was used to identify membrane proteins from a susceptible tumor cell line that preferentially bind to NK cells. Solubilized membrane proteins from surface biotin-labeled K562 cells were reacted with immunomagnetic bead enriched freshly isolated naïve NK cells or T lymphocytes. The membrane proteins from surface biotinylated K562 cells were reacted with freshly isolated viable CD3⁻, CD16⁺ enriched naïve NK cells, and CD3⁺, CD16⁻ enriched T lymphocytes. Reacted lymphocytes were washed and solubilized in Laemmli sample buffer. The proteins were then resolved by SDS-PAGE.

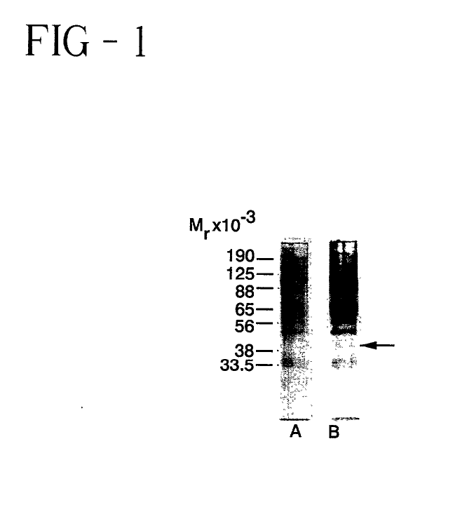

Lymphocyte bound biotinylated tumor plasma membrane proteins were detected on Western blots with streptavidin-alkaline phosphatase. The results are shown in FIG. 1 where lane A is a representative Western blot of surface labeled K562 membrane proteins that bound to enriched T lymphocytes and lane B is a representative Western blot of surface labeled K562 membrane proteins that bound to enriched NK cells. Densitometry analysis of the Western blots, employing Gelbase software (UVP's System 5000, Upland, Calif.), detected approximately 14 common bands of K562 plasma membrane proteins that bound to both NK cells and T lymphocytes. However, a single band of 38.5 kD (p38.5) was detected from NK cell adsorbed preparations but was not observed in T lymphocyte adsorbed preparations.

### Example 9

Binding of Purified P38.5 to Human Peripheral Blood Lymphocytes

Initial results suggested that p38.5 interacts with a receptor on the surface of naïve NK cells which was not expressed on T lymphocytes. Accordingly, p38.5 was purified by preparative SDS-PAGE to apparent homogeneity for additional studies.

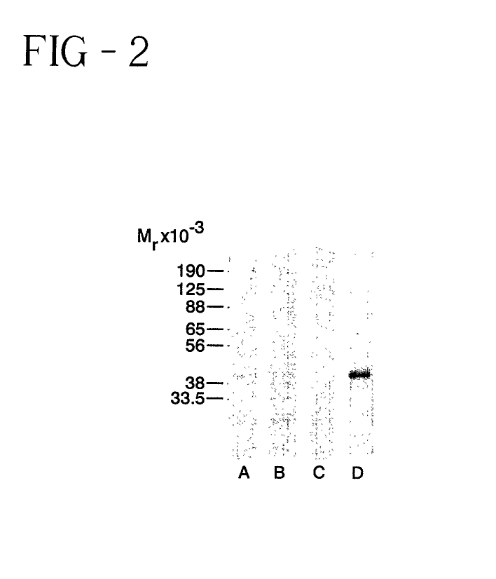

Binding reactions with purified p38.5 and detection of bound biotin labeled p38.5 were performed as described in Example 8. The results are shown in FIG. 2. Lane A is a Western blot of Coomassie blue-stained purified p38.5 K562 membrane protein. Lane B is a Western blot of an extract of viable HPBL that were reacted with purified biotin labeled p38.5 and shows slight reactivity. Lane C is a Western blot of an extract of viable enriched T lymphocytes that were reacted with purified biotin-labeled p38.5 and reveals little or no reactivity. Lane D is a Western blot of an extract of viable enriched NK cells that were reacted with purified biotin-labeled p38.5 demonstrating extensive reactivity. As shown in FIG. 2, lane A, the purified protein resolved as a single band of 38.5 kD molecular mass upon SDS-PAGE. For examining the binding properties of p38.5 to lymphocyte subsets, p38.5 was labeled with biotin, and the study carried out as described for proteins from the crude membrane preparation.

FIG. 2 shows that biotinylated p38.5 bound extensively to the NK cell enriched fraction (see FIG. 2, lane D), but did not bind at all to the enriched T lymphocytes (see FIG. 2, lane C). Unfractionated cells bound a slight amount of p38.5 (see FIG. 2, lane B) which is consistent with the low percentage of NK cells in peripheral blood lymphocyte specimens. These data also demonstrated that SDS-PAGE purified p38.5 retained its preferential binding property for naïve NK cells.

### Example 10

Structural Characteristics of p38.5

Initial attempts to obtain a partial amino acid sequence of the purified p38.5 were unsuccessful because the N-terminus of the molecule was blocked. Consequently, p38.5 was transferred onto nitrocellulose paper and subjected to in situ protease digestion as described in Aebersold et al., Proc. Natl. Acad Sci 84: 6970-6974 (1987). Amino acid sequence analysis of resultant peptides was performed at the Harvard Microchemistry Laboratory, Cambridge, Mass. An 11-mer amino acid sequence of an internal peptide (PheValAsnTrpGlnValAspGlyGluTyrArg : SEQ ID NO: 7) was obtained with a high degree of confidence. Comparison of this sequence with known sequences of proteins in GenBank did not reveal significant homology. Treatment of p38.5 with sulfhydryl reducing agents (2-mercaptoethanol and dithiothreitol) or deglycosylating enzymes (O-glycosidase, N-acetylneuraminidase II and peptide-N-glycosidase F, deglycosylation Kit, Glyko Inc., Novato, Calif.) did not alter the molecular mass of the molecule. This suggests that the isolated protein is a monomer and may not be glycosylated.

### Example 11

Preparation of Antibodies to P38.5

Antibody recognizing p38.5 was raised in a rabbit against an 11-mer synthetic peptide, PheValAsnTrpGlnValAspGlyGluTyrArg (SEQ ID NO: 7) derived from the amino acid sequence of an internal peptide of the molecule as described in Example 10. The synthetic peptide (250 μg) was mixed with Titermax (CytRx Co. Atlanta, Ga.) (1:1 v/v) and injected subcutaneously into a rabbit at 4 sites as instructed by the manufacturer. Subcutaneous booster injections (100 μg peptide in Freund's adjuvant incomplete) were given to the rabbit at bimonthly intervals. Serum was collected after one week following the third booster injection. Anti-p38.5 antibodies were affinity purified from sera using BSA-11 mer peptide conjugate linked to CNBr-Sepharose 4B.

Anti-25-mer antibody was made in rabbits against a 25-mer peptide: Ser Leu Gly Ala Gly Thr Ser Thr Ser Arg Ser Ser Glu Arg Thr Pro Gly Ala Ala Thr Ala Ser Ala Ser Gly (SEQ ID NO: 8) corresponding to amino acids 43-67 of SEQ ID NO: 2 in Table 2, in a similar manner as described for 11-mer antibody. Briefly, the synthetic peptide (250 ug) was mixed with Titermax (CytRx Co. Atlanta, Ga.) (1:1 vol/vol) and injected subcutaneous into a rabbit at four sites according to the manufacturer's instructions. Subcutaneous booster injections (100 ug peptide in IFA) were given at bimonthly intervals. Serum was collected 1 wk after the third booster injection and weekly thereafter. Both the antibodies were affinity purified from sera using CNBr-Sepharose 4B beads linked with BSA-peptide conjugate. Affinity purified antibody had a titer of 1:50,000 (25-mer) and 1:5,000 (11-mer) and reacted with a protein of 38.5 kD on Western blots (see below).

Both anti-11mer and anti-25mer antisera bound human p38.5 specifically. However, only the anti-11mer bound mouse p38.5, consistent with the conservation of the 11mer sequence in mouse p38.5 (See Table 4).

### Example 12

Expression of p38.5 on Established Tumor Cell Lines

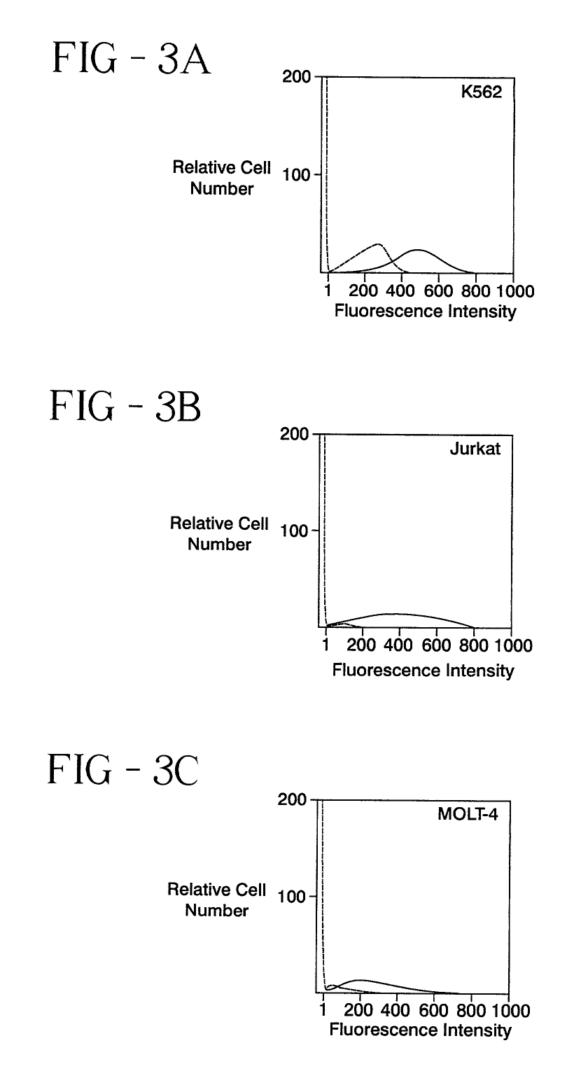

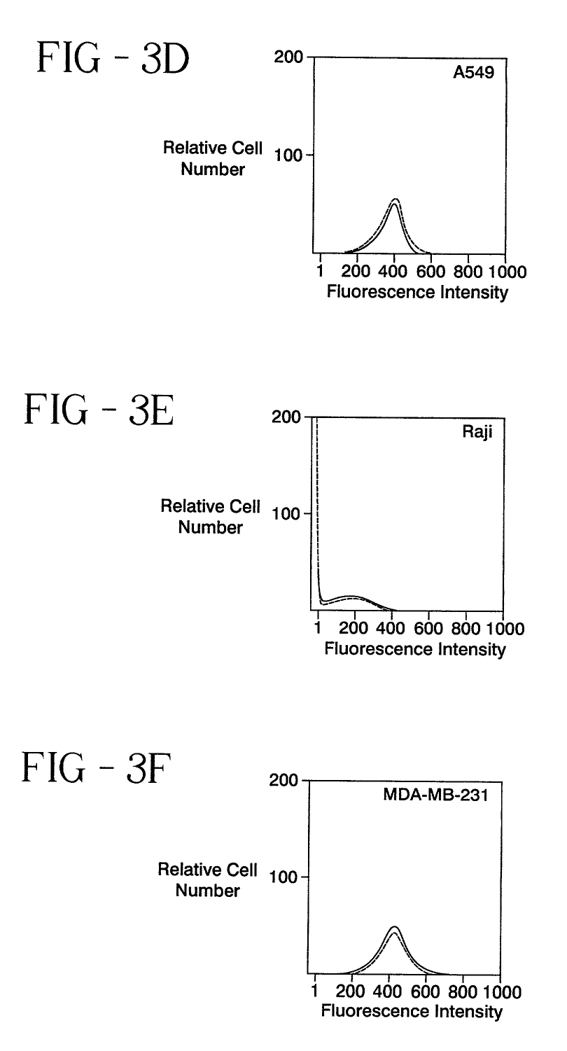

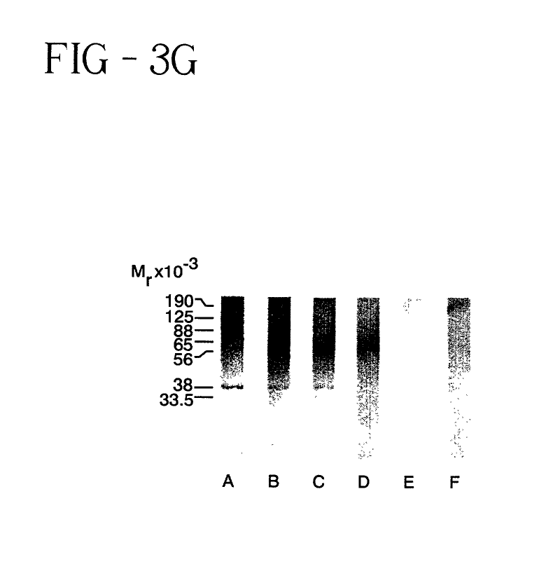

Since p38.5 (derived from K562 cells) exhibited selective binding to naïve NK cells, it was of interest to examine the expression of this molecule on the plasma membrane of NK resistant cell lines as well as other NK sensitive tumor cell lines. Affinity purified antibody raised against the internal 11 mer synthetic peptide of p38.5 (anti-p38.5) was employed for this purpose. Anti-p38.5 was reacted with three NK sensitive and three NK resistant tumor cell lines and analyzed separately by flow cytometry. The flow cytometry analysis of anti-p38.5 treated NK-susceptible and NK-resistant tumor cell lines is shown in FIG. 3A to FIG. 3F. Cells were treated with anti-p38.5 followed by goat anti-rabbit F(ab′)₂ IgG-FITC (____). Control tumor cells were treated with secondary antibody alone (----). Non-immune rabbit serum had similar reactivity as secondary antibody alone. As shown in FIG. 3A to FIG. 3F, anti-p38.5 reacted with the surface of NK sensitive tumor cell lines, K562 (erythroleukemia), Jurkat (T cell lymphoma) and Molt-4 (T cell leukemia). In contrast, anti-p38.5 did not bind to the three NK resistant tumor cell lines A549 (lung adenocarcinoma), Raji (Burkitt lymphoma, a B-cell lymphoma line) and MDA-MB231 (breast carcinoma).

Plasma membrane expression of p38.5 was also studied by anti-p38.5 immunoprecipitation of surface labeled (biotin) membrane proteins of the above tumor cells. FIG. 3G shows Western blots of anti-p38.5 immunoprecipitated proteins from surface biotinylated cells: K562 (lane A), Jurkat (lane B), Molt-4 (lane C), Raji, (lane D), A549 (lane E), and MDA-MB 231 (lane F). Proteins in these lanes were probed with streptavidin-alkaline phosphatase to detect cell surface biotinylated proteins. Biotin labeling and immunoprecipitation of plasma membrane proteins is described, for example, in Das et al. (1994). Analysis of the Western blots showed the presence of a single 38.5 kD biotinylated protein from NK-sensitive cell lines, whereas biotinylated proteins were not observed when the NK-resistant cell lines were employed. These results demonstrated an association between the expression of p38.5 on the surface of tumor cell lines and their susceptibility to NK cell-mediated lysis.

### Example 13

Cell Surface Expression of P38.5 on NK Resistant Variants of Jurkat and Molt-4 Cell Lines

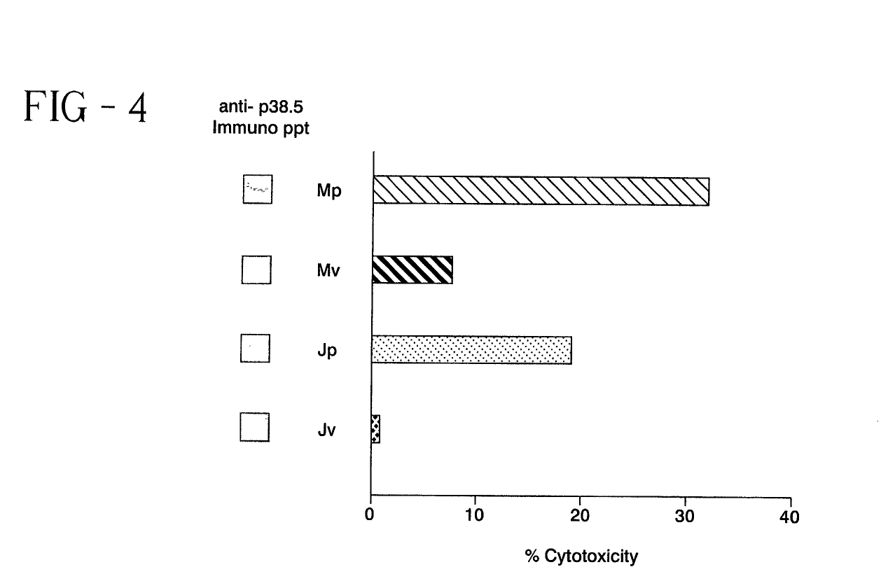

Though the expression of p38.5 is associated with susceptibility of well-established tumor cell lines to NK cell mediated cytolysis, one must interpret such data with caution as the cell lines are derived from different tissues and are likely to have been transformed by different mechanisms. This issue was addressed by examining the association of NK susceptibility and p38.5 expression in variants derived from wild type tumor cells. Following long term culture of Jurkat and Molt-4 cell lines, a substantial reduction in their susceptibility to NK cell-mediated cytotoxicity was observed. These variant cell lines were then analyzed for surface expression of p38.5 by anti-p38.5 immunoprecipitation. These results are shown in FIG. 4. Parental cell lines=Mp, Jp; Variant cell lines=Mv, Jv. Following long term culture of Jurkat (J) and Molt-4 (M) cell lines, substantial reduction in NK cell-mediated cytolysis was observed. These variant cell lines were analyzed for surface expression of p38.5 using a specific antibody as described above. Membrane proteins derived from 1×10⁶ cells were loaded in each lane.

The level of cytolytic activity was directly related to the apparent degree of expression of surface p38.5. Gelbase analysis (JVP, Upland Calif.) of the blots for protein bands from parental cell lines demonstrated relative single peak heights of 28 and 25, respectively. Similar analysis of the blots for protein bands from the variant cell lines did not reveal anti-p38.5 reactive protein. However, a very light band was visible by the naked eye (for Mv). The results shown in FIG. 4 demonstrated that variant cell lines exhibit decreased susceptibility to lysis by NK cells and express significantly lower amounts of p38.5 on their surface as compared to the wild type parental cell lines.

### Example 14

Effect of Purified P38.5 on NK Cell Mediated Cytolysis

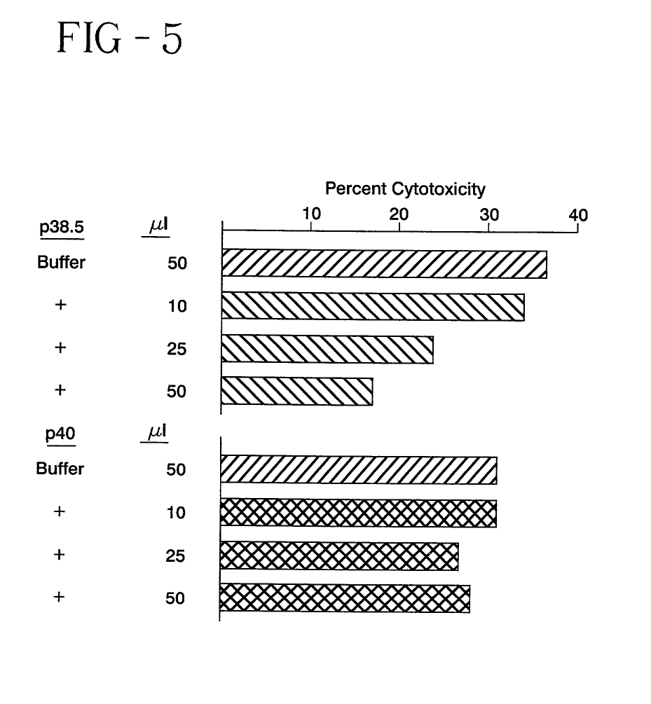

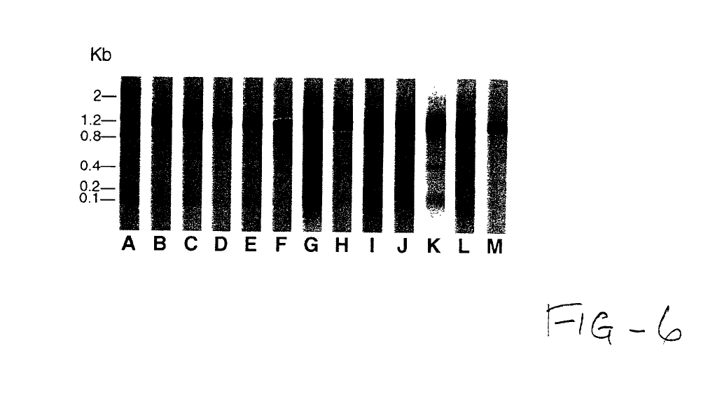

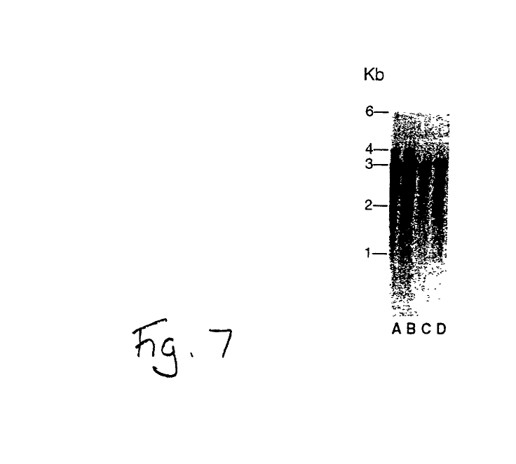

The preferential binding of p38.5 to NK cells suggested that the molecule may be involved as a ligand in the cytolytic process mediated by naïve NK cells. To directly examine this possibility, a purified preparation of p38.5 was incubated with human lymphocytes and then tested in a standard cytotoxicity assay against ⁵¹Cr-labeled K562 target cells. Incubation of lymphocytes with the purified p38.5 preparation inhibited naïve NK activity in a concentration dependent manner (see FIG. 5).

Human peripheral blood lymphocytes (HPBL) were pre-incubated with the indicated amount of purified proteins (approximately 45 ng/μl based on amino acid sequence data) at 37° C. for 30 minutes and then added to ⁵¹Cr-labeled K562 cells at an E/T ratio of 100:1 as previously described. Percent cytotoxicity was determined after 3 hours incubation by the release of ⁵¹Cr. See Das et al. (1994); Karpel and Norin (1989). Representative data of two experiments are shown in FIG. 5. The cytotoxicity data were analyzed by the Student's t test. The results obtained were p38.5, 10 μl, not significant; p38.5, 25 μl, p<0.01; p38.5, 50 μl, p<0.001 compared to the buffer control. p40 values compared to buffer control were not significant at any concentration.

These data provide direct evidence that interaction of the tumor surface protein p38.5 with its NK cell receptor is necessary for cytotoxicity since binding of soluble ligand to the NK cells prior to contact with K562 cells inhibited their lysis. A K562 membrane protein of 40 kD, purified by the same procedure as p38.5, did not affect NK activity.

### Example 15

Effect of anti-p38.5 antibody on naïve and IL-2 stimulated NK cell mediated cytotoxicity

The peptide CSKFVNWQVDGEYR (SEQ ID NO: 13) corresponding to amino acids 183 to 195 of p38.5 (including the sequence of the original 11mer peptide isolated from p38.5) was used to immunize rabbits. A cysteine residue was added at the N-terminus to facilitate covalent binding of the peptide to a solid matrix for subsequent affinity purification. Freshly isolated, non-cultured human lymphocytes were assayed for cytolytic activity. The cytotoxicity assay was performed against ⁵¹Cr-labeled K562 or A549 cells at a lymphocyte to target cell ratio of 100:1. K562 and A549 target cells were preincubated for 30 minutes with anti-peptide antibody prior to addition of lymphocytes. In parallel assays, Freshly isolated human lymphocytes were cultured for 6 days with IL-2 and then assayed for cytolytic activity as before. The results of these assays are shown in Table 5.

TABLE 5
Effect of anti-p38.5 antibody on naïve and IL-2 stimulated
NK cell mediated cytotoxicity

| PERCENT CYTOTOXICITY | |
|---|---|
| | ACTIVATED |
| NAÏVE NK CELLS | NK CELLS |

| ANTIBODY | EXPT. 1 | EXPT. 2 | | |
|---|---|---|---|---|
| DILUTION | K562 | K562 | K562 | A549 |
| 1:10 | 14 | 18 | 68 | 75 |
| 1:20 | 20 | 30 | 70 | 76 |
| 1:40 | 28 | 31 | 68 | 71 |
| 1:80 | 35 | nd | nd | nd |
| Buffer control | 34 | 49 | 70 | 73 |

nd Not done

The antibody treatment inhibited naïve NK cell mediate cytotoxicity(detected in K562 cells) but not cytotoxicity mediated by IL-2 stimulated NK cells (detected in A549 cells) in agreement with pervious data showing the presence of p38.5 on the surface of the former but not the latter cells.

### Example 16

Isolation of Total RNA and Polka RNA

Total cellular RNA was isolated using RNAqueous kit of Ambion (Austin, Tex.). Highly enriched polyA RNA was isolated using PolyA Pure mRNA purification kit following the manufacturers' instructions (Ambion, Austin, Tex.).

### Example 17

Reverse Transcriptase Polymerase Chain reaction (RT-PCR)

Expression of p38.5 mRNA was examined in normal and malignant cell lines using the Titan One Tube RT-PCR kit (Roche, Indianapolis, Ind.). High fidelity primers were selected using the Primer Selection Program (University of Minnesota) available at htt://alces.umn.edu/rawprimer.html.

A deletion mutant of p38.5 encoding cDNA was prepared for use in quantitative or semi-quantitative PCR assays. The mutant cDNA lacks the 128 base pairs between positions 402 and 530 of SEQ ID NO: 1 shown in Table 1.

A measured amount of the isolated and quantitated mRNA of the deletion mutant mRNA is spiked into the sample for real time PCR analysis. Primers are chosen that prime across the deletion. PCR products primed from the wild-type and deletion mutant cDNAs are of different lengths and may be resolved Comparison of the resultant signal intensity of PCR products primed from wild-type and deletion mutant targets allows estimation of the amount of p38.5 encoding cDNA originally present in the sample.

### Example 18

Preparation and labeling of RNA probes

Clone 70 was incubated with XhoI in a standard restriction enzyme digestion cocktail at 37° C. for 2 hours to linearize the cDNA. The anti-sense RNA probe was prepared from the linearized cDNA using MaxIscript in vitro transcription kit (Ambion, Austin, Tex.). The transcription reaction mixture contained 1 ug of DNA template, 2 ul transcription buffer, 10 mM ATP, GTP, UTP, 2 mM modified CTP and 2 ul T3 polymerase containing ribonuclease inhibitor. For the preparation of anti-sense RNA for actin probe T7 polymerase was used. The reaction mixture was incubated at 37° C. for 1 hour. Subsequently, 1 ul of DNAse I was added to the mixture and the content was incubated for an additional 15 min at 37° C. to destroy the template. To this mixture 20 ul of ammonium acetate and 1 vol of isopropyl alcohol were added, contents were mixed and placed at −20° C. for 15 min to precipitate the synthesized RNA probe. The precipitate was obtained by centrifugation at 30,000 g for 15 min and the pellet was washed once with 200 ul of 70% ethanol. Final pellet was dried in air and solubilized in 1×TE buffer. Labeling of the RNA probe was accomplished using Bright Star Psoralin-biotin kit of Ambion (Austin, Tex.). Appropriate amount of anti-sense RNA was mixed with TE and 4 ul of Psoralin biotin (provided in the kit) and irradiated for 45 min at ambient temperature in UV light at higher wavelength setting (approximately 365 nm). Unbound biotin was extracted twice with water saturated n-butanol. Labeled probe was either used fresh or stored at −80° C. before use.

### Example 19

5′ Rapid Amplification of cDNA Ends (RACE)

5′ primer extension was performed using the 5′-RACE kit of Gibco-BRL (Gaithersberg, Md.). Nested gene specific reverse primers were used in 3 consecutive amplifications: Primer 1, AAT GAC CTG AGC GTT GAG ACT (SEQ ID NO: 9), Primer 2, GGT AGT TGG ACT CCC CAT TGT (SEQ ID NO: 10), Primer 3, CGA TTG TGC TGA GGG CTA CT(SEQ ID NO: 11) each with the 5′ anchor primer, GCC CAC GCG TCG ACT AGT AC (SEQ ID NO: 12).

Poly A enriched RNA was used as template in the initial amplification. Human cDNA clone 5′R, (GenBank Accession number A316400, encoding amino acids 43-14 of p38.5) and human cDNA clone 5 (GenBank Accession number A316401, encoding amino acids 48-361 of p38.5) were isolated from K562 cells by this method.

### Example 20

Isolation and Seqencing of the cDNA Encoding Tumor Protein p38.5

cDNA libraries from two sources were used to screen for the p38.5 gene. A lambda-gt11 cDNA library constructed from the human erythroleukemia cell line (K562) was purchased from Clonetech (Pal Alto, Calif.), and a cDNA library of human lymhocytes that were depleted of monocytes, T cells and B cells, was constructed in the Zap Express vector using the Zap Express cDNA synthesis and cDNA Gigapack III Gold Cloning kits of Stratagene (La Jolla, Calif.).

Both the libraries were screened for putative p38.5 gene expression using the antibody raised against the p38.5 11-mer peptide. Positive clones from both the libraries were selected and amplified in suitable host bacteria prior to preparation of respective plasmids. Sequencing of the cloned cDNAs was performed at SUNY core sequencing facility and at Commonwealth Biotechnologies Inc. (Richmond, Va).

A clone designated 32 (GenBank Accession number AF316398) was isolated from a K562 lambda gt11 library and found to contain a cDNA fragment of 730 nucleotides encoding an ORF of 243 amino acids including 224 (amino acids 114-324 of SEQ ID NO: 2) from human p38.5 close to the C-terminal (See Table 2). Another clone, designated clone 70 (GenBank Accession number A316399) was isolated from a lambda Zap Express human lymphocyte library and contained a cDNA fragment of 1315 nucleotides encoding the 248 C-terminal amino acids from human p38.5 (See Table 2). Another 137 amino acids from the N-terminus of p38.5 was deduced from clones (5′R and 5: GenBank Accession numbers A316400 and 316401) obtained by primer extension using the 3′ and 5′ RACE kits of (GIBCO-BRL). A cDNA clone 402 (GenBank Accession number AF316402) likely carries the complete open reading frame (ORF) encoding human p38.5.

The predicted molecular weight of human p38.5 is 37.9kD. The nucleotide sequence of the assembled human p38.5 cDNA matches sequences in all exons of a gene on chromosome 19 (19q 13.2, GenBank Accession number AF050154) and the cDNA clone 402 matches an mRNA in GenBank, Accession number NM_{—}006114.

In recognition of the first published amino acid sequence data on p38.5 (Das et al., 1997), which was obtained from the human molecule on the surface of the K562 cell line as an eleven-mer peptide, the nomenclature “haymaker” was proposed. (HayMaKEr: human molecule on the surface of K562 cells reactive with antibody to an eleven-mer peptide).

Mouse cDNA clones were isolated using primers EC4 and RD1 (see Table 3) and the complete sequence of the mouse p38.5 ORF deduced (Table 4). The high GC content of both human and mouse coding sequences has made reverse transcription of these mRNAs and isolation of cDNA clones especially probematic. cDNA cloning required the use of the denaturant betain to allow efficient priming and extension reactions on these high GC templates by promoting melting of hairpin loop secondary structure.

### Example 21

DNA analysis by Southern Blot

Total cellular DNA for Southern analysis may be prepared by proteinase K digestion and subsequent phenol/chloroform extraction and ethanol precipitation. RNA is removed by digestion with 50 μg/ml RNase A. From each sample, 25 μg of DNA is digested with EcoRI and electrophoresed on a 0.8% agarose gel. The DNA on the gel is denatured, neutralized and transferred onto nitrocellulose (Schleicher and Schuell, Keene, N.H.) in 20×SSC overnight. The blot is baked, then blocked in hybridization solution composed of 50% formamide, 200 mM PIPES, 800 mM NaCl, 0.1% SDS, and 20 mg/ml blocking reagent (Boehringer Mannheim, Indianapolis, Ind.). Digoxigenin labeled oligonucleotide probes are labeled by random priming (using Prime-it II, Stratagene, LaJolla, Calif.), added to the hybridization solution and incubated at 42° C. overnight. The hybridized blots are washed at 42° C. hybridization temperature in 0.1×SSC/0.1% SDS and developed with anti-digoxigenin alkaline phosphatase.

### Example 22

Expression of p38.5 mRNA in Malignant and Non-malignant Cells

Northern analysis of p38.5 gene transcription was performed with polyA-enriched RNA using the Northern Max kit of Ambion (Austin, Tex.). Approximately 0.8 ug of polyA-enriched RNA from cells was mixed with loading buffer, heated at 65° C. for 30 min and subjected to 1% agarose gel electrophoresis. RNA from the gel was transferred to Bright Star-plus positively charged nylon membrane (Ambion, Austin, Tex.) by capillary action. Membranes containing transferred RNA were washed for 10 sec with running buffer, dried in air and cross-linked by incubating at 80° C. for 15 min.

Subsequently, the membrane was incubated in the prehybridzation buffer at 65° C. for 30 min followed by in hybridization buffer containing approximately 3 ug of biotinylated anti-sense clone 70 RNA probe overnight at the same temperature. The membrane was successively washed twice with washing buffer, stringent washing solution and incubated with blocking solution for 30 min at ambient temperature. The membrane was incubated with streptavidin-alkaline phosphatase for 30 min, washed several times and incubated for 5 min with chemiluminiscent substrate (CDP-star). The membrane was soaked in TE buffer, wrapped in plastic and incubated in the dark overnight. The hybridized probe was detected on X-ray films by standard procedures.

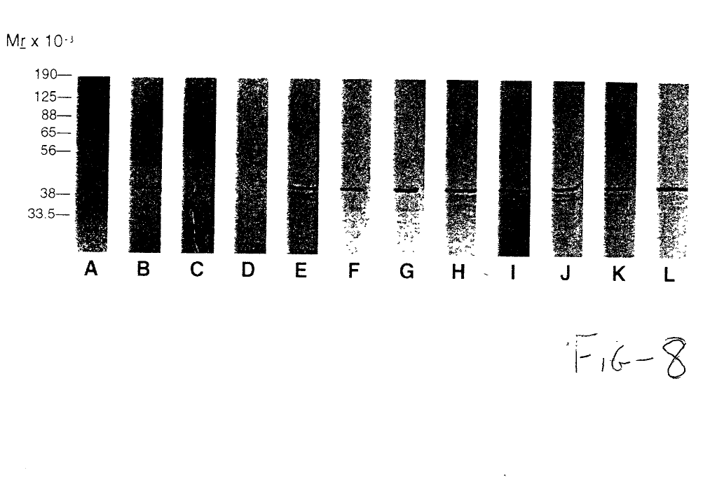

In one experiment mRNA was isolated from A549, K562, CD16 enriched HPBLs (NK cells) and CD3 enriched HPBLs (T cells) and resolved on agarose gel and blotted (FIG. 6). The amount of polyA RNA loaded onto the agarose gel was approximately 800 ng for each cell type. The blot was hybridized with a biotinylated RNA probe derived by transcription of clone 70 and developed using CDP Bright Star developer (Ambion Inc). FIG. 6, Lane A, human adenocarcinoma cell line (A549); lane B, human erythroleukemia cell line (K562); lane C, CD16 enriched HPBL (NK cells); lane D, CD3 enriched HPBL (T-cells).

One hybridized product estimated to be approximately 3 Kb was observed in all three cell types. Since the ORF is 1.1 Kb and the 3′ untranslated region is 0.4 Kb, the 5′ untranslated region is unusually long, extending approximately 1.5 Kb.

### Example 23

Reverse Transcriptase PCR (RT-PCR) Analysis of Malignant and Non-malignant Cell Lines

Total RNA from different cell lines was subjected to RT-PCR using p38.5 gene specific primers (LP-1; CTA CCA CTT CGG GGT CAC AT and XF-2 CTC CTC CCT GAG OTT GTC TG). Amplified products were resolved on agarose gel containing ethidium bromide and localized under UV light.

In FIG. 7 the lanes were loaded as follows: Lane A, DNA size markers; lane B, human peripheral blood lymphocytes; lane C, human endothelial cell line (HUVEC); lane D, human embryonic fibroblast cell line (CCD-27SK); lane E, a non-malignant cell line; lane F, human erythroleukemia cell line (K562); lane G, human lymphoma (Jurkat); lane H, human lymphoma (Molt-4); lane I, human lung adenocarcinoma (A549); lane J, human breast carcinoma (T47-D); lane K, human breast carcinoma (MD231); lane L, human kidney carcinoma (DU145); lane M, human colorectal carcinoma (COLO). A single product of expected size (1017 bp) was observed for all cell lines. This amplicon spans from a portion of exon 3 into exon 9 (AF050154).

In order to rule out the amplification of an irrelevant mRNA, the cDNA product obtained by using RNA from the CCD-27SK human fibroblast cell line and HPBL were sequenced. Each of the nucleotide sequences was identical to the sequence obtained from the cDNA derived by RT-PCR of mRNA from the K562 cell line.

Both Northern analysis (Example 22) and RT-PCR analysis (this Example) indicate, therefore, that the p38.5 gene is transcribed in all cells, including non-malignant cells and tumor cells that do not exhibit surface localization of the protein.

### Example 24

Detection of p38.5 by Immunoblot Analysis of Cell Extracts of Malignant and Non-malignant Cell Lines

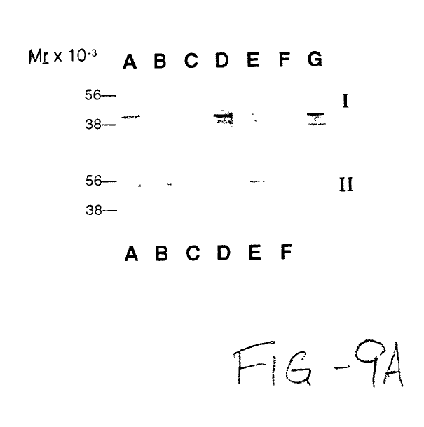

Immuno-blot analysis was performed on whole cell extracts of p38.5 surface-negative and surface-positive cells using the anti-25-mer antibody (generated against amino acids 43-67 of SEQ ID NO: 2). A 38.5 kD band was detected in 5 representative carcinoma cell lines (p38.5 surface-negative) as well as three representative hematopoeitic tumors cell lines (p38.5 surface-positive) as shown in FIG. 8.

Whole cell extracts from different cell lines were separated by SDS-PAGE and transblotted. Extracts from 2×10⁵ cells were loaded into each of the lanes of the gel. Blots were reacted with anti-25-mer followed by goat anti-rabbit alkaline phosphatase. A major band of 38.5 kD was detected in all tumor cell lines. Non-malignant cells with the exception of the HUVEC line did not exhibit a 38.5 kD reactive band. In FIG. 8, the lanes were loaded as follows: lane A: HPBL; lane B: human endothelial cell line, HUVEC; lane C: human embryonic fibroblast, CCD 27-SK; lane D: a non-malignant cell line; lane E: human erythroleukemia cell line, K562; lane F: human lymphoma Jurkat; lane G: human lymphoma Molt-4; lane H: human lung adenocarcinoma, A549; lane I: human breast carcinoma, T47-D; lane J: human breast carcinoma, MD231; lane K: human kidney carcinoma, DU145; lane L: human colorectal carcinoma, COLO.

Obvious quantitative differences in the expression of p38.5 in the whole cell extracts of the latter two types of malignant cell lines were not observed suggesting that cell surface expression does not directly correlate with the quantity of intracellular p38.5. Importantly, p38.5 was not detected in whole cell extracts of normal cells and non-malignant cell lines including; HPBL, CCD-27SK, a human fibroblast cell line and a non-malignant cell line. A human umbilical cord endothelial cell line (HUVEC) was slightly reactive (FIG. 8). These data clearly indicate that p38.5 is present in an intracellular compartment at substantially greater levels in cells of malignant origin compared to cells of non-malignant origin.

### Example 25

Western Blot Analyses of p38.5 and F₁β-ATPase in Whole Cell Extracts and Particulate Fractions of Malignant and Non-malignant Cells

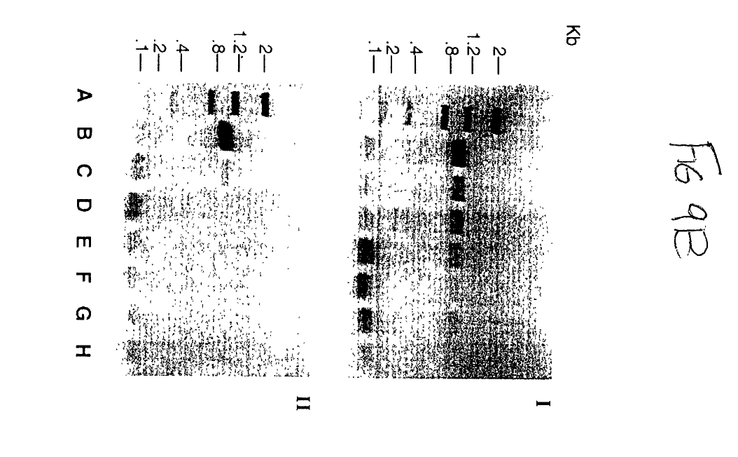

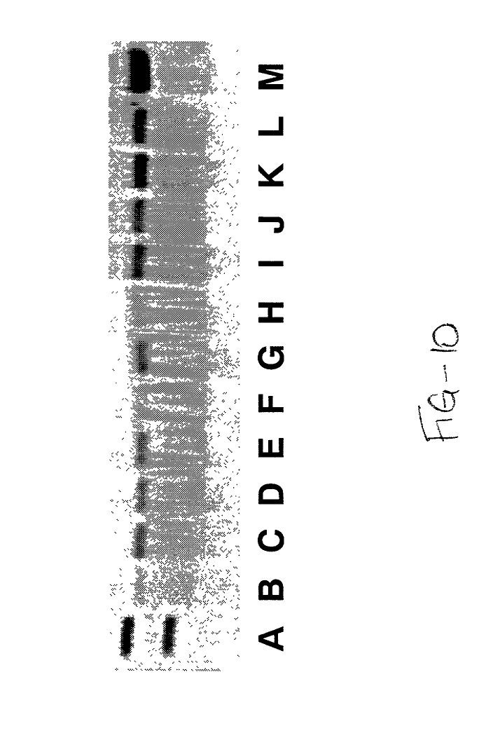

Whole cell extracts of K562, human peripheral blood lymphocytes (PBLs) and of the human fibroblast cell line CCD-27SK were compared with 30,000 g particulate fractions (including membranes) from each. Samples were trans-blotted following SDS-PAGE. Lysate from 2.10⁵ cells were loaded into each of the lanes. Blots were reacted either with anti-25 mer (FIG. 9A, panel I) or anti-F₁βATPase (FIG. 9A, panel II). Immunoblots developed as described in Example 24. Lane A, whole cell extract of K562; lane B, whole cell extract of HPBL; lane C, whole cell extract of human fibroblast CCD-27SK cell line; lane D, 30,000 g membrane fragment of K562; lane E, 30,000 g fraction of PBL; lane F, 30,000 g fraction of CCD-27SK; lane G, 10×concentration of 30,000 g fraction of CCD-27SK cell line. Similar reactivity is seen with the anti-F₁βATPase but differential reactivity is revealed with anti-p38.5 antibody.

The blots shown in FIG. 9A reveal a 38.5 kD protein in the 30,000 g pellet as well as whole cell extracts of K562 cells. Supernatants of pelleted extracts were not reactive. A 38.5 kD band was neither observed in the whole cell extracts nor in the 30,000 g pellet of HPBL or CCD-27SK even though the gels were loaded with material from the equivalent number of cells. A 38.5 kD band was detected, however, on 10 fold concentration of the 30,000 g pellet of CCD-27SK, thereby demonstrating a low level of the 38.5kD protein in these non-malignant cells. In contrast to anti-p38.5 reactivity, the mitochondrial marker (anti-F1βATPase, Das et. al, 1994) was detected on immuno-blots of both non-malignant cells and malignant cell lines from the above extracts at approximately the same intensity. The lack of concordant association of the levels of a standard mitochondrial marker with the levels of p38.5 in malignant and non-malignant cells suggests that the latter protein does not localize to mitochondria.

Confocal photomicroscopy of immunofluorescence staining of A549 cells, a naïve NK resistant tumor cell line shows non-uniform, particulate intracellular staining by anti p38.5 antibody. Staining is in a compartment that is distinct from mitochondria, as double label immunofluorescent staining with anti-mitochondrial protein antibody further demonstrates that the two antibodies do not coincide in distribution.

### Example 26

Semi-quantitative Analysis of p38.5 Gene Transcription in a Malignant and a Non-malignant Cell Line

Above data demonstrate the detection of a substantial quantity of intracellular p38.5 in malignant cell lines whereas little protein is detected in non-malignant cells (FIG. 8) even though p38.5 specific mRNA is detected in the latter (FIGS. 6 and 7). To further examine p38.5 gene transcription, a semi-quanitative RT-PCR procedure was performed with template RNA from K562 and the non-malignant cell line of Example 24 and FIG. 8 (i.e. comparing a cell that expresses a high level of protein to a cell line that expresses a low level of the protein, respectively). Serial dilutions of the two templates were amplified separately using standard forward and reverse gene specific primers.

RT-PCR was performed with total RNA from K562, (FIG. 9B, panel II) and a non-malignant cell line (FIG. 9B, panel II). Serial four-fold dilutions of RNA were used in RT-PCR reactions with the same primer set, LP-1 and XF2, as in FIG. 6. Lane A=DNA size markers, lane B=1 ng RNA, lane C=250 pg RNA, lane D=62.5 pg RNA, lane E=15.6 pg RNA, lane F=4.4 pg RNA, lane G=1.1 pg RNA, lane H=0.25 pg RNA. Amplified product was resolved on agarose gel containing ethidium bromide and localized by UV light. A decreasing intensity of p38.5 cDNA was observed with K562 RNA (1 ng to 1 pg), no product was observed at 0.25 pg K562 RNA. The non-malignant cell line revealed an amplified product with 1 ng and 250 pg of RNA only.

A 1017 bp amplicon was observed when the quantity of the nucleic acid from either source was equal to or greater than 250 pg (FIG. 9B, lanes B and C). A gene specific amplicon was not observed when the template from the non-malignant cell line was diluted to less than 60 pg (FIG. 9B, Panel II, lanes D to H). In another experiment with the same nonmalignant cell line, the 1017 bp amplicon was detected with 100 pg of total RNA but not with 10 pg of total RNA. In contrast, RT-PCR of total RNA from K562 cells yielded the 1017 bp amplicon when the amount of template was diluted to >10 pg (FIG. 9B, Panel I, lane G). These data reveal that the erythroleukemia malignant cell line, K562 expresses about 10 fold more p38.5 mRNA than the non-malignant cell line.

Examination of the Northern blot shown in FIG. 6 supports and extends the results of the above RT-PCR experiment. Both malignant cell lines, K562 and A549 had similar amounts of p38.5 specific message that was substantially greater than the amount of specific mRNA from T lymphocyte enriched HPBL or NK cell enriched HPBL. A substantial difference in the relative abundance of p38.5 transcripts in malignant cell lines compared to non-malignant cells correlates with a the greater level of detectable protein in cancer cells (FIG. 8). Concentration of protein extracts by 10 fold permitted detection of p38.5 in antibody probed Western Blots of non-malignant cell lines. Taken together these data suggest transcriptional up-regulation of p38.5 synthesis in cancer cells.

### Example 27

Lymphocyte Stimulation Induces p38.5 Transcripts

Human T lymphocytes were stimulated with two different polyclonal mitogens, Phorbol-12-Myristate 13-acetate (PMA), 7.5 ng/ml, and Phytohemagglutinin-P (PHA), 3 ug/ml, each purchased from Sigma Chemical Co., St. Louis, Mo. T cells were incubated in RPMI medium at 37° C. After the specified period cells were removed from the culture, enumerated and total RNA was isolated. p38.5 primers LP-1 and XF-2 and 100 ng of RNA were used in each RT-PCR reaction. RT-PCR products were resolved by agarose gel electrophoresis and visualized with ethidium bromide, as shown in FIG. 10: Lane A: Size markers, upper band at 1.2 kb and the lower band at 0.8 kb. Lane B: RNA from unstimulated T lymphocytes; FIG. 10: Lanes C, E, G, I and K show RT-PCR products from T lymphocytes stimulated with PMA for 16, 24, 40, 48 and 72 hours, respectively. FIG. 10: Lanes D, F, H, J and L show RT-PCR products from T lymphocytes stimulated with PHA for 16, 24, 40, 48 and 72 hours, respectively. FIG. 10: Lane M: RT-PCR amplicon of approximately 1.0 kb from RNA of the unstimulated K562 tumor cell line.

The level of p38.5 specific transcripts is clearly shown to increase during stimulation with an activating agent (such as a phorbol ester, PMA or a mitogenic lectin, PHA) indicating that activated lymphocytes can be detected by methods that quantitate p38.5-specific RNA expression. Conditions, disorders and diseases in which activated lymphocyte interactions may play a role include organ and tissue transplantation (including bone marrow), infectious diseases and autoimmune diseases. Therefore, methods for the detection of p38.5-specific transcripts as exemplified above, may be a valuable diagnostic or monitoring technique in these situations.

### Example 28

Expression of Full Length p38.5 in E. coli

Clone 402 encoding the full length open reading frame (ORF) of p38.5 was expressed in E.coli. The ORF coding sequence was cloned in the pET-38(+) vector purchased from Novagen. This vector has a cellulose binding domain (CBD) tag and a 6×His tag in fusion at the C-terminus of p38.5. A signal sequence at the N-terminus causes the recombinant protein to be exported to the periplasm of the bacteria for increased solubility. Antibody to the CBD tag was shown to react with the p38.5 construct in Western Blots.

### Example 29

Production of Fusion Protein or Peptides of p38.5

The pET-32 LIC vector or other suitable vectors such as Baculovirus based vectors (Novagen, Madison, Wis.) are used according to the manufactures instructions. Full length p38.5 is used as template for PCR. Primers are designed according to the Novagen manual and contain a short vector overhang sequence linked by an ATX triplet to a gene specific oligonucleotide. Various fragments of the cDNA or a full length cDNA are cloned into the vector by appropriate selection of primers to produce fusion proteins (peptides of different length). The PCR product is eluted from an agarose gel and ligated to the pET-32 vector. The plasmid is purified by a plasmid isolation kit (Qiagen) and used to transform competent E. coli supplied in the Novagen kit. Expression of recombinant proteins is by incubation of log phase E.coli with IPTG. The His tagged fusion protein is then purified by affinity on a Ni column. Fusion protein production is verified by SDS-PAGE and Western blot analysis. Fusion proteins/peptides of p38.5 are used as immunogens for production of polyclonal and monoclonal antibodies.

### Example 30

Characterization of NK Cell Receptor of p38.5

In order to further delineate the preferential interaction of p38.5 with NK cells relative to T lymphocytes, we conducted investigations to identify an interactive molecular species on effector cells. In direct binding assays, biotin labeled purified p38.5 was reacted with either NK cell or T lymphocyte membrane proteins which had been previously immobilized on Western blots. A Western blot of Coomassie blue stained purified p38.5 K562 membrane protein; T lymphocyte proteins (Bound p38.5 was not detected on this blot indicating the absence of reactive T lymphocyte proteins); and NK cell proteins. Labeled p38.5 was detected at approximately 70 kD in the K562 membrane protein preparation and in the total NK cell proteins, demonstrating a preferential interaction with the latter immobilized NK cell protein. The reactivity of p38.5 with the 70 kD NK cell protein was markedly reduced when an extensively denatured (by boiling) preparation of NK cell proteins was employed. Labeled p38.5 was detected as a single band at 70 kD on blots of NK cell membrane proteins, whereas reactive bands were not detected on blots of T lymphocyte membrane proteins. This result suggests that a protein of 70 kD from naïve NK cells binds cell surface p38.5 from NK cell-susceptible tumors and that the 70 kD protein is either not present or is present in substantially lower amounts in T lymphocytes.

### Example 31

Immunologic Identification of p38.5 Bound to p70

To establish the immunologic identity of the ligand bound to the NK cell receptor, anti-p38.5 serum was employed. A Western blot of peripheral blood lymphocyte membrane proteins was initially incubated with a solubilized crude extract of K562 membrane proteins, washed and then reacted with anti-p38.5 and developed with goat anti-rabbit alkaline phosphatase.

A Western blot of K562 membrane protein extract and HPBL membrane proteins was reacted with affinity purified anti-p38.5 and revealed the absence of anti-p38.5 reactive proteins. A Western blot of HPBL membrane proteins reacted with K562 membrane extracts for 2 h at ambient temperature, washed and probed with anti-p38.5 detected a band at an approximate molecular weight of 70 kD.

Western blots of lymphocyte membrane proteins that were not pre-incubated with the tumor proteins did not react with anti-p38.5. These results suggest that the 38.5 kD surface molecule from K562 cells which binds to the 70 kD naive NK cell protein contains the 11 mer peptide to which antibody was raised. It can be concluded from these results that the 11-mer epitope of p38.5 is probably not directly involved in the receptor-ligand interaction. (Presumably if the p38.5-70 kD interaction included the 11 mer epitope of p38.5, antibody to the latter determinant could not bind to the complex).

### Example 32

Plasma Membrane Localization of p70

The following experiment was performed to confirm that a 70 kD NK cell protein (p70) that specifically binds p38.5 is localized on the plasma membrane. Solubilized membrane proteins from surface biotinylated naïve NK cells were applied to a p38.5-Sepharose-4B affinity column. After extensive washing with 0.5 M NaCl buffer to remove non-specifically bound NK cell membrane proteins, the remaining specifically bound proteins were eluted by solubilization of the beads with Laemmli sample buffer (denaturing buffer), resolved by SDS-PAGE, and subjected to Western blot analysis. p38.5-Sepharose 4B bound NK cell membrane proteins were detected on Western blots using standard streptavidin-alkaline phosphatase reaction.

The following samples were included: Lane A: crude extract of NK cell membrane proteins from surface biotinylated NK cells prior to affinity chromatography. Lane B: cell membrane protein(s) specifically bound to the p38.5 affinity column. A single discrete biotinylated band of 70 kD from NK cells was detected on the blots suggesting that this p38.5 binding protein (p70) is located on the exterior of the plasma membrane (the cell surface). One possibility is that the 70 kD, p38.5 binding protein is the inducible form of heat shock protein (Hsp 70). We performed flow cytometry studies utilizing antibody to Hsp 70 (Stressgen Biotech. Corp. Victoria, BC Canada, Clone C92F3A-5) to determine surface expression of this molecule on T and NK cells. These experiments indicated that neither NK nor T cells expressed Hsp70 on their plasma membranes in contrast to K562 cells which served as a positive control.

Similarly, this assay may be used in screening to identify compounds which block specific binding of molecules (such as p70) to p38.5.

### Example 33

Construction of NK Cell cDNA Library

Construction of cDNA library of highly enriched naïve NK cells was achieved using the following methods.

a. Isolation of NK Cells

Human peripheral blood lymphocytes (HPBL) were fractionated to enrich for NK cells. Non-adherent HPBL were obtained after overnight incubation at 4 C on a plastic disk. These non-adherent HPBL were subjected to two cycles of nylon wool column adsorption to obtain lymphoctes further depleted of B cells and monocytes. These lymphocytes were then treated with anti-HLA class II immunomagnetic beads. Negative cells (i.e., those that did not bind to the beads) were treated with anti-CD5 and anti-CD3 (Becton-Dickenson, San Jose, Calif.) in succession to remove T lymphocytes utilizing goat anti-mouse IgG immunomagnetic beads (Dynal Inc., Lake Success, N.Y.). This negatively selected lymphocyte preparation (class II³¹ , CD3⁻, and CD5⁻, and enriched for CD16⁺ NK cells) was utilized for construction of the cDNA library. The positively selected T cells were used for RNA subtraction.

b. Purification of NK Cell mRNA by Subtraction Hybridization.

mRNA from naive NK cells (HLA class II³¹ , CD3³¹ CD5³¹ , CD16⁺) was isolated utilizing an mRNA isolation kit (Stratagene, La Jolla, Calif.). NK cell mRNA was then utilized to make single strand cDNA (Stratagene cDNA library kit), whereas T cell mRNA was photobiotinylated following instructions provided in the subtraction kit (Invitrogen, San Diego, Calif.). NK cDNA and T cell biotinylated mRNA were hybridized (for 36 hours) and the hybridization mixture was treated with streptavidin and extracted to remove hybridized cDNA-mRNA and free mRNA. See the Invitrogen subtraction kit manual for further details. Hybridization was repeated once more to ensure complete removal of contaminating biotinylated hybridized T cell mRNA and free mRNA. Unique NK cell cDNA (in aqueous phase) was further processed as described below.

c. Preparation of cDNA Library.

Subtracted NK cell cDNA obtained in the procedure described in Example 17 b was utilized to clone in Lambda ZaP Express using the Gigapack III Gold Cloning kit according to the instruction manual (Stratagene, La Jolla, Calif.). Briefly, single strand NK cell cDNA was utilized to synthesize the second strand. The termini of the double-stranded cDNA were filled in with Pfu DNA polymerase and EcoR I adapters were ligated to the blunt ends. Subsequently, EcoRI adapter and residual linker-primer from the 3′ end of the cDNA were released by Xho1 digestion. Fragments were then separated on a Sephacryl S 500 spin column. The size fractionated cDNA was then precipitated and ligated to the Lambda Zap Express vector arms. The Lambda library was packaged in a high-efficiency system of Gigapack III Gold packaging extract and finally the library was plated on the E. Coli cell line XL1-Blue MRF′.

d. Screening of the library for NK Cell p70.

The strategy for isolation of clones that express p70 was to identify those plaques which bind p38.5 and therefore acquire reactivity for anti-p38.5 antibodies. Plaques of XL1-Blue MRF infected with λ phage were transferred to nitrocellulose paper. After appropriate blocking with 5% BSA, the paper was initially treated with the solubilized extract of K562 membrane protein (containing the p38.5 ligand) washed and then incubated with anti-p38.5 antibodies. Anti-p38.5 reactive plaques were identified by reaction with biotinylated goat anti-rabbit IgG followed by streptavidin alkaline phosphatase and NBT/BCIP mixture. Positive plaques were purified further by secondary and tertiary screening. Positive reaction of plaques without incubation with K562 membrane protein served as a control.

e. Sequencing of Positive Inserts.

Purified positive plaques were used in an in vivo excision protocols utilizing Exassist helper phage and XLOLR E. coli strain. This procedure enables one to obtain colonies containing a PBK-CMV double-stranded phagemid vector with the cloned DNA insert (details of the procedure are given in the Strategene Instruction manual). XLOLR strain of such phagemid vector were then utilized to isolate the plasmid using a plasmid isolation kit supplied by Qiagen (Chatsworth, Calif.).

### Example 34

Preparation of Monoclonal Antibodies

Monoclonal antibodies may be generated by subcutaneous (SC) injection of mice with p38.5 or p38.5 fragments. Such immunogens may be generated as fusion proteins/polypeptides, as described above in Example 29. The adjuvant and immunization schedule is similar to that as described for polyclonal antibodies. The clone cell-HY Monoclonal Antibody Production Kit (Stem Cell Technologies, Inc., Vancouver, BC Canada) may be used according to the manufacturer's instructions. Spleen cells from immunized mice are isolated and fused to mouse myeloma cells by the polyethylene glycol method. Fused and isolated cells are incubated 16 to 24 hours at 37° C. in 5% CO₂ in recovery medium. The cells are then washed and placed into semisolid methylcellulose-HAT-based selection medium and plated into petri dishes. Monoclonal colonies arise in 10-14 days and are picked up with a pipette and transferred to individual wells in a 96-well tray. The supernatants from the wells are tested for the desired antibody using either ELISA, Western blot, or flow cytometry. The desired hybridomas are expanded in growth medium. Large quantities of high titer antibodies may be produced by the ascities method. See, for example, Current Protocols in Immunology, Coligan et al., eds., John Wiley & Sons (1991).

### Example 35

Treatment of Autoimmune Reactions that Involve NK Cells

NK cells may play a role in autoimmune reactions, for example diabetes, rheumatoid diseases and multiple sclerosis, and additionally in the rejection of allogeneic and xenogeneic organs, tissues and cells by direct cytolytic interaction with target cells or by the release of cytokines. The activity of NK cell may be altered by interaction with specific antibodies to the receptor for the p38.5 or by competitive inhibition with soluble ligand (p.38.5). See Das et al. J Exp. Med. 185:1735 (1997).

Thus, while there have been described what are presently believed to be the preferred embodiments of the present invention, those skilled in the art will realize that other and further embodiments may be made without departing from the spirit of the invention, and it is intended to include all such further modifications and changes as come within the true scope of the claims set forth herein.

What is claimed is:

1. A method for diagnosing a tumor cell, comprising: obtaining a sample comprising a cell from a subject, and
(a) determining the level of p38.5 expressed at the cell surface; or
(b) determining the total p38.5 expressed by the cell;
wherein a higher than normal level of p38.5 expressed at the cell surface or a higher than normal level of total p38.5 expressed by the cell is indicative of a tumor cell.

2. The method according to claim 1, wherein the level of p38.5 expressed at the cell surface, or the total p38.5 expressed by the cell is determined by binding with an antibody that specifically binds p38.5.

3. The method according to claim 2, wherein the antibody that specifically binds p38.5 is bound by a second antibody.

4. The method according to claim 3, wherein the second antibody is conjugated to a detectable moiety.

5. The method according to claim 4, wherein the detectable moiety is a chromophore, a fluorescent compound, an enzyme, a polymer bead, a magnetic bead or a member of a specific binding pair.

6. The method according to claim 1, wherein the level of p38.5 expressed at the cell surface, or the total p38.5 expressed by the cell is determined by enzyme-linked immunosorbent assay (ELISA), radioimmunoassay (RIA), scintillation proximity (SPA), immunoprecipitation, Immunofluorescence, immunoelectrophoresis, immunodiffusion or agglutination.

7. The method according to claim 2, wherein the antibody specifically binds an epitope having an amino acid sequence found within SEQ ID NO: 2.

8. The method according to claim 7, wherein the epitope is found within an amino acid sequence from position 43 to position 67 or from position 185 to 195 of SEQ ID NO: 2.

9. The method according to claim 1, wherein the cell is a mammalian cell.

10. The method according to claim 9, wherein the mammalian cell a human cell.

11. The method according to claim 9, wherein the cell is sensitive to lysis by naïve NK cells.

12. The method according to claim 9, wherein the cell is from a an organ or tissue selected from the group consisting of brain, heart, lung, breast, kidney, liver, thyroid, esophagus, larynx, stomach, intestine, colorectal tissue, eye, skin, muscle, blood, pancreas, bone, ovary, testicle, prostate, bladder, endometrium, soft tissue, neuronal tissue and adipose tissue.

13. The method according to claim 9, wherein the cell sensitive to killing by naïve NK cells.

14. The method according to claim 13, wherein the cell is a hematopoietic cell, a lung cell, a breast cell, a kidney cell or a colorectal cell.

15. The method according to claim 1, wherein the amino acid sequence of p38.5 is at least 90% identical to SEQ ID NO: 2.

16. The method according to claim 15, wherein the amino acid sequence of p38.5 is at least 95% identical to SEQ ID NO: 2.

17. The method according to claim 16, wherein the amino acid sequence of p38.5 is at least 97% identical to SEQ ID NO: 2.

18. The method according to claim 17, wherein the amino acid sequence of p38.5 is at least 99% identical to SEQ ID NO: 2.

19. The method according to claim 18, wherein the amino acid sequence of p38.5 is the sequence of SEQ ID NO: 2.

20. The method according to claim 1, wherein the cell is an intact cell and wherein the level of p38.5 expressed by the cell is determined.

21. The method according to claim 1, wherein the cell is a lysed cell and wherein the total p38.5 expressed by the cell is determined.

22. A method for diagnosing a tumor cell, comprising: obtaining a sample comprising
a cell from a subject, and
determining the level of p38.5-specific RNA present in the cell;
wherein a higher than normal level of p38.5-specific RNA in the cell is indicative of a tumor cell.

23. The method according to claim 22, wherein the p38.5-specific RNA comprises a nucleotide sequence at least 90% identical to the transcript of SEQ ID NO.: 2.

24. The method according to claim 23, wherein the p38.5-specific RNA comprises a nucleotide sequence at least 95% identical to the transcript of SEQ ID NO.: 2.

25. The method according to claim 24, wherein the p38.5-specific RNA comprises a nucleotide sequence at least 97% identical to the transcript of SEQ ID NO.: 2.

26. The method according to claim 25, wherein the p38.5-specific RNA comprises a nucleotide sequence at least 99% identical to the transcript of SEQ ID NO.: 2.

27. The method according to claim 26, wherein the p38.5-specific RNA comprises a nucleotide sequence of the transcript of SEQ ID NO.: 2.

28. The method according to claim 22, wherein the level of p38.5-specific RNA is determined by reverse transcriptase-polymerase chain reaction (RT-PCR).

29. The method according to claim 28 wherein the reverse transcriptase polymerase chain reaction (RT-PCR) is quantantitative RT-PCR.

30. The method according to claim 29 wherein the quantantitative reverse transcriptase polymerase chain reaction (RT-PCR) is real time quantantitative reverse transcriptase polymerase chain reaction.

31. The method according to claim 28, wherein the reverse transcriptase polymerase chain reaction (RT-PCR) comprises priming from at least two primers; and wherein at least one primer comprises a sequence suitable for priming from the nucleic acid sequence of SEQ ID NO.: 2 and at least one primer comprises a sequence suitable for priming from the complement of the nucleic acid sequence of SEQ ID NO.: 2.

32. The method according to claim 22, wherein the level of RNA encoding p38.5 present in the cell is determined by hybridization.

33. The method according to claim 32, wherein the hybridization comprises hybridization of a probe that hybridizes with the nucleotide sequence of SEQ ID NO.: 2, or the complement of the nucleotide sequence of SEQ ID NO.: 2.

34. The method according to claim 22, wherein the cell is a mammalian cell.

35. The method according to claim 34, wherein the mammalian cell is a human cell.

36. A method of monitoring a disorder, disease or condition associated with a higher than normal level of expression of p38.5 in a subject, the method comprising:
providing two or more cell samples taken at different times from a subject to be tested; and
(c) determining the level of cell surface p38.5 in each of the cell samples, and comparing the level of cell surface p38.5 in each of the cell samples; or
(d) determining the level of total p38.5 in each of the cell samples, and comparing the level of total p38.5 in each of the cell samples; thereby monitoring the disease, disorder or condition.

37. A method of monitoring a disorder, disease or condition associated with a higher than normal level of expression of p38.5-specific RNA in a subject, the method comprising: providing two or more cell samples taken at different times from a subject to be tested, determining the level p38.5-specific RNA in each of the cell samples, and comparing the level p38.5-specific RNA in each of the cell samples, thereby monitoring the disease, disorder or condition.

38. A method of treating a subject suffering from a tumor associated with cell surface expression of p38.5, comprising administering an effective amount of an antibody that specifically binds p38.5.

39. A method for identifying a compound as an inhibitor of natural killer (NK) cell mediated killing of cells that express a higher than normal level of cell surface p38.5, comprising:
(i) providing a first sample of cells that expresses a higher than normal level of cell surface p38.5;
(ii) contacting the first sample of cells with the test compound;
(iii) contacting the first sample of cells with naïve NK cells, and
(iv) assessing the NK cell mediated cytotoxicity in the first sample of cells;
(v) providing a second sample of cells that expresses a higher than normal level of cell surface p38.5, identical to the first sample of cells;
(vi) contacting the second sample of cells with naïve NK cells, and
(vii) assessing the NK cell mediated cytotoxicity in the second sample of cells;
(viii) comparing the NK cell mediated cytotoxicity in the first sample of cells with the NK cell mediated cytotoxicity in the second sample of cells;
wherein a lower NK cell mediated cytotoxicity assessed in the first sample of cells than assessed in the first sample of cells identifies the compound as an inhibitor of natural killer (NK) cell mediated killing of cells that express p38.5 at their cell surface.

40. A method for determining the number of natural killer (NK) cells in a biological sample, comprising:
(a) contacting the sample containing NK cells with an antibody comprising a detectable label moiety and having specific binding affinity for p70 protein, under conditions permissive for binding of the antibody to p70 protein expressed by NK cells in said sample; and
(b) counting the number of cells bound by said antibody, thereby permitting determination the number of natural killer cells in the sample.
